SEQUENCE LISTING (129 sequences)

SEQ ID NO: 1 - protein, 243 aa, Arabidopsis thaliana
MIGQLMNLKATELCLGLPGGAEAVESPAKSAVGSKRGFSETVDLMLNLQSNKEGSVDLKNVSAVPKEKTTLKDPSKPPAKAQVVGWPPVRNYRKNMMTQQKTSSGAEEASSEKAGNFGGGAAGAGLVKVSMDGAPYLRKVDLKMYKSYQDLSDALAKMFSSFTMGNYGAQGMIDFMNESKLMNLLNSSEYVPSYEDKDGDWMLVGDVPWEMFVESCKRLRIMKGSEAVGLAPRAMEKYCKNRS

SEQ ID NO: 2 - protein, 189 aa, Arabidopsis thaliana
MDEFVNLKETELRLGLPGTDNVCEAKERVSCCNNNNKRVLSTDTEKEIESSSRKTETSPPRKAQIVGWPPVRSYRKNNIQSKKNESEHEGQGIYVKVSMDGAPYLRKIDLSCYKGYSELLKALEVMFKFSVGEYFERDGYKGSDFVPTYEDKDGDWMLIGDVPWEMFICTCKRLRIMKGSEAKGLGCGV

SEQ ID NO: 3 - protein, 229 aa, Arabidopsis thaliana
MMGSVELNLRETELCLGLPGGDTVAPVTGNKRGFSETVDLKLNLNNEPANKEGSTTHDVVTFDSKEKSACPKDPAKPPAKAQVVGWPPVRSYRKNVMVSCQKSSGGPEAAAFVKVSMDGAPYLRKIDLRMYKSYDELSNALSNMFSSFTMGKHGGEEGMIDFMNERKLMDLVNSWDYVPSYEDKDGDWMLVGDVPWPMFVDTCKRLRLMKGSDAIGLAPRAMEKCKSRA

SEQ ID NO: 4 - protein, 228 aa, Arabidopsis thaliana
MNLKETELCLGLPGGTETVESPAKSGVGNKRGFSETVDLKLNLQSNKQGHVDLNTNGAPKEKTFLKDPSKPPAKAQVVGWPPVRNYRKNVMANQKSGEAEEAMSSGGGTVAFVKVSMDGAPYLRKVDLKMYTSYKDLSDALAKMFSSFTMGSYGAQGMIDFMNESKVMDLLNSSEYVPSYEDKDGDWMLVGDVPWPMFVESCKRLRIMKGSEAIGLAPRAMEKFKNRS

SEQ ID NO: 5 - protein, 163 aa, Arabidopsis thaliana
MANESNNLGLEITELRLGLPGDIVVSGESISGKKRASPEVEIDLKCEPAKKSQVVGWPPVCSYRRKNSLERTKSSYVKVSVDGAAFLRKIDLEMYKCYQDLASALQILFGCYINFDDTLKESECVPIYEDKDGDWMLAGDVPWEMFLGSCKRLRIMKRSCNRG

SEQ ID NO: 6 - protein, 321 aa, Arabidopsis thaliana
MSYRLLSVDKDELVTSPCLKERNYLGLSDCSSVDSSTIPNVVGKSNLNFKATELRLGLPESQSPERETDFGLLSPRTPDEKLLFPLLPSKDNGSATTGHKNVVSGNKRGFADTWDEFSGVKGSVRPGGGINMMLSPKVKDVSKSIQEERSHAKGGLNNAPAAKAQVVGWPPIRSYRKNTMASSTSKNTDEVDGKPGLGVLFVKVSMDGAPYLRKVDLRTYTSYQQLSSALEKMFSCFTLGQCGLHGAQGRERMSEIKLKDLLHGSEFVLTYEDKDGDWMLVGDVPWEIFTETCQKLKIMKGSDSIGLAPGAVEKSKNKERV

SEQ ID NO: 7 - protein, 13 aa, Arabidopsis thaliana
QVVGWPPVRNYRK

SEQ ID NO: 8 - protein, 13 aa, Arabidopsis thaliana
QVVGWPPVRSYRK

SEQ ID NO: 9 - protein, 13 aa, Arabidopsis thaliana
QIVGWPPVRSYRK

SEQ ID NO: 10 - protein, 13 aa, Arabidopsis thaliana
QIVGWPPIRSYRK

SEQ ID NO: 11 - protein, 13 aa, Arabidopsis thaliana
QVVGWPPIRSYRK

SEQ ID NO: 12 - protein, 13 aa, Arabidopsis thaliana
QVVGWPPIRSFRK

SEQ ID NO: 13 - protein, 13 aa, Arabidopsis thaliana
QVVGWPPVCSYRR

SEQ ID NO: 14 - protein, 13 aa, Arabidopsis thaliana
QAVGWPPVCSYRR

SEQ ID NO: 15 - protein, 13 aa, Arabidopsis thaliana
QVVGWPPVRSYRR

SEQ ID NO: 16 - protein, 4 aa, Arabidopsis thaliana
NMMT

SEQ ID NO: 17 - protein, 4 aa, Arabidopsis thaliana
NIMT

SEQ ID NO: 18 - protein, 4 aa, Arabidopsis thaliana
NIIT

SEQ ID NO: 19 - protein, 4 aa, Arabidopsis thaliana
NVMA

SEQ ID NO: 20 - protein, 4 aa, Arabidopsis thaliana
NIMA

SEQ ID NO: 21 - protein, 4 aa, Arabidopsis thaliana
SVMA

SEQ ID NO: 22 - protein, 4 aa, Arabidopsis thaliana
TVMA

SEQ ID NO: 23 - protein, 4 aa, Arabidopsis thaliana
NVMV

SEQ ID NO: 24 - protein, 4 aa, Arabidopsis thaliana
NMMV

SEQ ID NO: 25 - protein, 4 aa, Arabidopsis thaliana
NVMG

SEQ ID NO: 26 - protein, 4 aa, Arabidopsis thaliana
NVLV

SEQ ID NO: 27 - protein, 4 aa, Arabidopsis thaliana
NNIQ

SEQ ID NO: 28 - protein, 4 aa, Arabidopsis thaliana
NNVQ

SEQ ID NO: 29 - protein, 4 aa, Arabidopsis thaliana
NNIH

SEQ ID NO: 30 - protein, 4 aa, Arabidopsis thaliana
NTMA

SEQ ID NO: 31 - protein, 4 aa, Arabidopsis thaliana
NTMS

SEQ ID NO: 32 - protein, 4 aa, Arabidopsis thaliana
KNSL

SEQ ID NO: 33 - protein, 4 aa, Arabidopsis thaliana
KNSF

SEQ ID NO: 34 - protein, 7 aa, Arabidopsis thaliana
NMMTQQK

SEQ ID NO: 35 - protein, 7 aa, Arabidopsis thaliana
NIMTQQK

SEQ ID NO: 36 - protein, 7 aa, Arabidopsis thaliana
NIMTNQK

SEQ ID NO: 37 - protein, 7 aa, Arabidopsis thaliana
NIITQQK

SEQ ID NO: 38 - protein, 7 aa, Arabidopsis thaliana
NVMANQK

SEQ ID NO: 39 - protein, 7 aa, Arabidopsis thaliana
NIMANQK

SEQ ID NO: 40 - protein, 7 aa, Arabidopsis thaliana
SVMAHQK

SEQ ID NO: 41 - protein, 7 aa, Arabidopsis thaliana
TVMATQK

SEQ ID NO: 42 - protein, 7 aa, Arabidopsis thaliana
NVMAQPK

SEQ ID NO: 43 - protein, 8 aa, Arabidopsis thaliana
NVMVSCQK

SEQ ID NO: 44 - protein, 8 aa, Arabidopsis thaliana
NMMVSCQK

SEQ ID NO: 45 - protein, 8 aa, Arabidopsis thaliana
NVMGSCQK

SEQ ID NO: 46 - protein, 8 aa, Arabidopsis thaliana
NVLVSSQK

SEQ ID NO: 47 - protein, 8 aa, Arabidopsis thaliana
NVMGSYQK

SEQ ID NO: 48 - protein, 7 aa, Arabidopsis thaliana
NMMVAQK

SEQ ID NO: 49 - protein, 7 aa, Arabidopsis thaliana
NNIQSKK

SEQ ID NO: 50 - protein, 7 aa, Arabidopsis thaliana
NNIQTKK

SEQ ID NO: 51 - protein, 7 aa, Arabidopsis thaliana
NNVQTKK

SEQ ID NO: 52 - protein, 7 aa, Arabidopsis thaliana
NNIQIKK

SEQ ID NO: 53 - protein, 7 aa, Arabidopsis thaliana
NNIHTKK

SEQ ID NO: 54 - protein, 9 aa, Arabidopsis thaliana
NTMASSTSK

SEQ ID NO: 55 - protein, 8 aa, Arabidopsis thaliana
NTMASSSK

SEQ ID NO: 56 - protein, 9 aa, Arabidopsis thaliana
NTMASNPSK

SEQ ID NO: 57 - protein, 9 aa, Arabidopsis thaliana
NTMATNPSK

SEQ ID NO: 58 - protein, 9 aa, Arabidopsis thaliana
NTMAANPSK

SEQ ID NO: 59 - protein, 9 aa, Arabidopsis thaliana
NTMSSQSSK

SEQ ID NO: 60 - protein, 9 aa, Arabidopsis thaliana
NTMASNPPK

SEQ ID NO: 61 - protein, 9 aa, Arabidopsis thaliana
NTMAPNPSK

SEQ ID NO: 62 - protein, 9 aa, Arabidopsis thaliana
NTMASNSAK

SEQ ID NO: 63 - protein, 9 aa, Arabidopsis thaliana
NTMANNSSK

SEQ ID NO: 64 - protein, 8 aa, Arabidopsis thaliana
KNSLERTK

SEQ ID NO: 65 - protein, 8 aa, Arabidopsis thaliana
KNSLEQTK

SEQ ID NO: 66 - protein, 8 aa, Arabidopsis thaliana
KNSFERTK

SEQ ID NO: 67 - protein, 17 aa, Arabidopsis thaliana
QVVGWPPVRNYRKNMMT

SEQ ID NO: 68 - protein, 17 aa, Arabidopsis thaliana
QVVGWPPVRNYRKNIMT

SEQ ID NO: 69 - protein, 17 aa, Arabidopsis thaliana
QVVGWPPVRNYRKNIIT

SEQ ID NO: 70 - protein, 17 aa, Arabidopsis thaliana
QVVGWPPVRNYRKNVMA

SEQ ID NO: 71 - protein, 17 aa, Arabidopsis thaliana
QVVGWPPVRNYRKNIMA

SEQ ID NO: 72 - protein, 17 aa, Arabidopsis thaliana
QVVGWPPVRNYRKSVMA

SEQ ID NO: 73 - protein, 17 aa, Arabidopsis thaliana
QVVGWPPVRNYRKTVMA

SEQ ID NO: 74 - protein, 17 aa, Arabidopsis thaliana
QVVGWPPVRSYRKNIMA

SEQ ID NO: 75 - protein, 17 aa, Arabidopsis thaliana
QVVGWPPVRSYRKNVMV

SEQ ID NO: 76 - protein, 17 aa, Arabidopsis thaliana
QVVGWPPVRSYRKNMMV

SEQ ID NO: 77 - protein, 17 aa, Arabidopsis thaliana
QVVGWPPVRSYRKNVMG

SEQ ID NO: 78 - protein, 17 aa, Arabidopsis thaliana
QVVGWPPVRSYRKNVLV

SEQ ID NO: 79 - protein, 17 aa, Arabidopsis thaliana
QIVGWPPVRSYRKNNIQ

SEQ ID NO: 80 - protein, 17 aa, Arabidopsis thaliana
QIVGWPPIRSYRKNNIQ

SEQ ID NO: 81 - protein, 17 aa, Arabidopsis thaliana
QIVGWPPVRSYRKNNVQ

SEQ ID NO: 82 - protein, 17 aa, Arabidopsis thaliana
QIVGWPPVRSYRKNNIH

SEQ ID NO: 83 - protein, 17 aa, Arabidopsis thaliana
QIVGWPPVRSYRKNSIQ

SEQ ID NO: 84 - protein, 17 aa, Arabidopsis thaliana
QVVGWPPIRSYRKNTMA

SEQ ID NO: 85 - protein, 17 aa, Arabidopsis thaliana
QVVGWPPIRSYRKNTMS

SEQ ID NO: 86 - protein, 17 aa, Arabidopsis thaliana
QVVGWPPIRSFRKNTMA

SEQ ID NO: 87 - protein, 17 aa, Arabidopsis thaliana
QVVGWPPVCSYRRKNSL

SEQ ID NO: 88 - protein, 17 aa, Arabidopsis thaliana
QAVGWPPVCSYRRKNSL

SEQ ID NO: 89 - protein, 17 aa, Arabidopsis thaliana
QVVGWPPVRSYRRKNSF

SEQ ID NO: 90 - DNA, 306 bp, Artificial Sequence
  AtIAA17 Codon optimized cDNA sequence
aagcggggcttcagcgagaccgtggacctgaagctgaacctgaacaatgagcccgccaataaggagggctccaccacacacgacgtggtgacatttgattctaaggagaagagcgcctgccctaaggaccccgcaaagccacctgccaaggcacaggtggtgggatggccacccgtgcggtcctacagaaagaacgtgatggtgtcttgtcagaagagctccggcggccccgaggcagcagccttcgtgaaggtgtctatggacggcgccccttacctgaggaagatcgatctgcgcatgtataag

SEQ ID NO: 91 - DNA, 336 bp, Artificial Sequence
  AtIAA7 cDNA codon optimized
aagaggggcttctctgagaccgtggacctgatgctgaacctgcagtccaataaggagggctctgtggatctgaagaacgtgagcgccgtgcctaaggagaagaccacactgaaggacccatccaagccccctgccaaggcacaggtggtgggatggccacccgtgcggaactacagaaagaatatgatgacccagcagaagacaagctccggcgcagaggaggcatctagcgagaaggccggcaattttggaggaggagcagcaggagcaggactggtgaaggtgtccatggacggagcaccatacctgcggaaggtggatctgaagatgtataag

SEQ ID NO: 92 - DNA, 309 bp, Artificial Sequence
  AtIAA14 cDNA codon optimized
aagaggggcttctctgagaccgtggacctgaagctgaacctgcagagcaataagcagggccacgtggatctgaacaccaatggcgcccctaaggagaagacatttctgaaggacccaagcaagccccctgccaaggcacaggtggtgggatggccacccgtgcggaactacagaaagaatgtgatggccaaccagaagtccggcgaggcagaggaggcaatgagctccggcggaggcaccgtggccttcgtgaaggtgtctatggacggagcaccatacctgcggaaggtggatctgaagatgtataca

SEQ ID NO: 93 - DNA, 234 bp, Artificial Sequence
  AtIAA3 cDNA codon optimized
aagcgggtgctgtccaccgacacagagaaggagatcgagagctcctctaggaagaccgagacatccccacctaggaaggcacagatcgtgggatggccacccgtgcggtcttacagaaagaacaatatccagagcaagaagaacgagtccgagcacgagggccagggcatctatgtgaaggtgtctatggacggcgccccctacctgaggaagatcgatctgagctgctataag

SEQ ID NO: 94 - protein, 308 aa, Artificial Sequence
  Degradation signal peptide (aas 37-104 of AtIAA7 (SEQ ID NO 1)) fused to N-terminus of mEGFP via a two-AA linker (SG)
GFSETVDLMLNLQSNKEGSVDLKNVSAVPKEKTTLKDPSKPPAKAQVVGWPPVRNYRKNMMTQQKTSSSGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 95 - protein, 238 aa, Artificial Sequence
  mEGFP
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 96 - protein, 575 aa, Arabidopsis thaliana
MNYFPDEVIEHVFDFVTSHKDRNAISLVCKSWYKIERYSRQKVFIGNCYAINPERLLRRFPCLKSLTLKGKPHFADFNLVPHEWGGFVLPWIEALARSRVGLEELRLKRMVVTDESLELLSRSFVNFKSLVLVSCEGFTTDGLASIAANCRHLRDLDLQENEIDDHRGQWLSCFPDTCTTLVTLNFACLEGETNLVALERLVARSPNLKSLKLNRAVPLDALARLMACAPQIVDLGVGSYENDPDSESYLKLMAVIKKCTSLRSLSGFLEAAPHCLSAFHPICHNLTSLNLSYAAEIHGSHLIKLIQHCKKLQRLWILDSIGDKGLEVVASTCKELQELRVFPSDLLGGGNTAVTEEGLVAISAGCPKLHSILYFCQQMTNAALVTVAKNCPNFIRFRLCILEPNKPDHVTSQPLDEGFGAIVKACKSLRRLSLSGLLTDQVFLYIGMYANQLEMLSIAFAGDTDKGMLYVLNGCKKMKKLEIRDSPFGDTALLADVSKYETMRSLWMSSCEVTLSGCKRLAEKAPWLNVEIINENDNNRMEENGHEGRQKVDKLYLYRTVVGTRMDAPPFVWIL

SEQ ID NO: 97 - protein, 582 aa, Morus notabilis
MASSFPEEVLEHVFSFIQSDADRNSISMVCKSWYEIERWCRRRIFVGNCYAVSPRMVIRRFPDVRSIELKGKPHFADFNLVPEGWGGYVDPWISAMAMAYPWLEEIRLKRMVVTDESLELIAKSFKNFKALVLSSCEGFSTDGLAAIAANCRNLRELDLRESDVDDLSGHWLSHFPDTYTSLVSLDISCLGSEMSFTALERLVGRCPNLRSLRLNRTVPIEKLANLLRQAPQLVELGTGAYSAELRPEVFSNLAGALSGCKELKSLSGFWEAVPAYLPAVYSICPGLTSLNLSYAAIQSPHLIKLLSHCPKLQRLWVLDYIEDVGLEALAASCKDLRELRVFPSDPFVAEPNVSLTEQGLVSVSGGCPKLHSVLYFCRQMSNAALTTIARNRPNFTCFRLCIIEPRTPDYLTLGPLDAGFGAIVEHCKDLRRLSVSGLLTDRAFEYIGTYAKKLEMLSLAFAGDSDLGLHHVLSGCENLRKLEIRDCPFGDKALLANAAKLETMRSLWMSSCMVSYGACKLLGQKMPRLNVEVIDERGPPDTRPESCPVEKLYIYRSVAGPRFDMPGFVWTMDEDSSALRLS

SEQ ID NO: 98 - protein, 572 aa, Gossypium hirsutum
MNYFPDEVLEHVFDFITSHKDRNSVSLVCKSWYKIERCSRQRVFIGNCYSISPERLIARFPGLKSLTLKGKPHFADFNLVPHDWGGFVYPWIEALAKSRIGLEELRLKRMVVSDESLELLSKSFVNFKSLVLVSCEGFTTDGLAAIAANCRFLRELDLQENEVDDHRGHWLSCFPESCTSLISLNFACLRGEVNLGALERLVSRSPNLKSLRLNRAVPLDTLQKLLMRAPQLVDLGIGSYVHDPFSEAYNKLKIAIQRCKSIRSLSGFLEVAPHCMSAIYPICGNLTFLNLSYAPGLHGNKLMKLIQHCRKLQRLWILDCIGDKGLGVVALTCKELQELRVFPSDPFEAGNAAVTEEGLVLVSAGCPKLNSLLYFCQQMTNAALITVAKNCPNFIRFRLCILDPIKPDPVTNQPLDEGFGAIVQSCKGLKRLSLSGLLTDQVFLYIGMYAEQLEMLSIAFAADSDKGMLYVLNGCKKLRKLEIRDCPFGDAALLEDVGKYETMRSLWMSSCEVTLGGCKSVAEKMPSLNVEIIDESEQMEFNLDDKQKVDKMYLYRTLVGHRKDAPEYVWIL

SEQ ID NO: 99 - protein, 575 aa, Noccaea caerulescens
MNYFPDEVTEQVFDFVTSHKDRNAISLVCKSWYKIERYSRQRVFIGNCYAINPERLLRRFPCLKSLTLKGKPHFADFNLVPHEWGGFLQPWIEALARSHVGLEELRLKRMVVTDESLELLSRSFSNFKSLVLVSCEGFTTDGLASIAANCRHLRDLDLQENEIDDHRGQWLSCFPDTCTSLVTLNFACLEGETNLVALERLVARSPKLKSLKLNRAVPLDALARLVACAPQIVELGVGSYENDRESEQYLKLEAAIKKCTSLRSLSGFLEAAPHSLSAFHPICHNLTSLNLSYAAEIHSSHLIKLIQHCNKLQRLWILDSIGDKGLEVVASTCKELQELRVFPSDLVGGGNTAVTEDGLVAISAGCPKLHSILYFCQQMTNAALITVAKNCPNFIRFRLCILEPNKPDHVTSRPLDEGFGAIVRACKRLRRLSLSGLLTDQVFHYIGKYAHELEMLSIAFAGDTDKGMLYVLDGCKKMRKLEIRDSPFGDAALLADVSKYETMRSLWMSSCEVTLGGCKRIAQIAPWLNVEIINENDNNRMEQNGHEGRQEVDKLYLYRTVVGTRTDAPPFVWIL

SEQ ID NO: 100 - protein, 573 aa, Morus notabilis
MNYFPDEVIEHVFDYVTTHKDRNALSLVCKSWYRIERLSRLRVFIGNCYAISPERTVERFPGLRSLTLKGKPHFADFNLVPHEWGGFVFPWIEALARSRVALEELRLKRMVVSDESLELLSRSFANFKSLVLVSCEGFTTDGLAAIAANCRHLRELDLQENEIEDNRGQWLSCFADNCTSLVSLNFACLKGEINLAALERLVARSPDLRVLRVNRAVPLDALQKILMKAPQLVDLGTGSYVTDSRSDAYNKLKSTLLKCQSIRSLSGFLEVVPWCLPAFYPVCSNLTSLNLSYAPGIYGFELIKLIRHCVKLQRLWILDCIGDKGLSVVASTCKELQELRVFPSDPYGLGHAAVTEEGLVAISVGCPKLHSLLYFCQQMTNAALITVAKNCPNFIRFRLCILDPTKPDPVTSQPLDEGFGAIVQACKSLRRLSLTGLLTDQVFLYIGMYAEQLEMLSVAFAGDSDKGMLYVLRGCKKLRKLEIRDCPFGDVALLTDVGKYETMRSLWMSSCEVTLGACKTVARKMPSLNVEIINEHDQTEFCLDDDEQKVEKMYLYRTTVGPRTDAPEFVWTL

SEQ ID NO: 101 - DNA, 1725 bp, Artificial Sequence
  AtAFB2 cDNA codon optimized
atgaactactttcccgatgaggtcattgagcacgtctttgatttcgtcacaagccacaaggataggaacgccattagcctggtctgtaagagctggtacaagatcgagcggtattccagacagaaggtgttcatcggcaactgctacgccatcaatccagagaggctgctgcggagatttccctgtctgaagagcctgaccctgaagggcaagccccacttcgccgactttaacctggtgcctcacgagtggggaggattcgtgctgccatggatcgaggccctggcacggtccagagtgggcctggaggagctgaggctgaagcgcatggtggtgacagacgagtctctggagctgctgtctcggagcttcgtgaacttcaagagcctggtgctggtgtcctgcgagggctttaccacagatggcctggcaagcatcgcagcaaactgtaggcacctgcgggacctggacctgcaggagaatgagatcgacgatcacagaggccagtggctgtcctgcttccccgatacctgtaccacactggtgacactgaactttgcctgcctggagggcgagacaaatctggtggccctggagaggctggtggcacgctctcccaacctgaagagcctgaagctgaatagggcagtgcctctggacgcactggcaagactgatggcatgcgcaccacagatcgtggatctgggcgtgggctcctacgagaacgaccctgattccgagtcttatctgaagctgatggccgtgatcaagaagtgtaccagcctgcgcagcctgtccggcttcctggaggcagcacctcactgcctgtccgcctttcacccaatctgtcacaacctgacatccctgaatctgtcttacgccgccgagatccacggctcccacctgatcaagctgatccagcactgcaagaagctgcagaggctgtggatcctggactccatcggcgataagggcctggaggtggtggcctctacctgtaaggagctgcaggagctgcgcgtgttcccatctgacctgctgggaggaggaaacaccgcagtgacagaggagggcctggtggcaatctctgccggatgccccaagctgcacagcatcctgtatttttgtcagcagatgaccaacgccgccctggtgacagtggccaagaactgccccaatttcatcaggtttcgcctgtgcatcctggagcccaataagcctgaccacgtgacatctcagcctctggatgagggcttcggcgccatcgtgaaggcctgcaagagcctgaggcgcctgtctctgagcggcctgctgaccgaccaggtgttcctgtacatcggcatgtatgccaaccagctggagatgctgtctatcgcctttgccggcgacacagataagggcatgctgtacgtgctgaatggctgtaagaagatgaagaagctggagatcagagacagcccttttggcgataccgccctgctggcagacgtgagcaagtatgagacaatgcggagcctgtggatgagctcctgcgaggtgaccctgagcggctgtaagagactggccgagaaggccccatggctgaacgtggagatcatcaacgagaatgacaacaataggatggaggagaatggccacgagggccgccagaaggtggataagctgtatctgtataggaccgtcgtcgggactcggatggatgcccccccctttgtctggattctg

SEQ ID NO: 102 - DNA, 1746 bp, Artificial Sequence
  MnTIR1 cDNA codon optimized
atggccagctccttccccgaggaggtgctggagcacgtgttctcttttatccagagcgacgccgataggaactctatcagcatggtgtgcaagagctggtacgagatcgagcggtggtgccggagaaggatcttcgtgggcaattgttatgccgtgagcccaaggatggtcatccgccggtttcctgacgtgagatccatcgagctgaagggcaagccacacttcgccgactttaacctggtgccagagggatggggaggatacgtggatccttggatctccgccatggccatggcctatccctggctggaggagatcagactgaagaggatggtggtgaccgacgagtctctggagctgatcgccaagagcttcaagaacttcaaggccctggtgctgtctagctgcgagggcttttctacagatggactggcagcaatcgcagcaaactgtaggaatctgcgggagctggacctgcgcgagagcgatgtggacgatctgagcggccactggctgtcccacttccctgacacctacacatccctggtgtctctggatatctcctgcctgggcagcgagatgtcctttaccgccctggagaggctggtgggccgctgtcctaacctgaggtctctgaggctgaatcgcacagtgccaatcgagaagctggccaacctgctgaggcaggcaccacagctggtggagctgggaaccggagcatatagcgccgagctgagaccagaggtgttctccaatctggcaggcgccctgtctggatgcaaggagctgaagtccctgtctggcttttgggaggcagtgccagcatacctgcctgccgtgtattccatctgtcccggcctgacatccctgaacctgtcttacgccgccatccagtctccacacctgatcaagctgctgagccactgcccaaagctgcagcggctgtgggtgctggactatatcgaggatgtgggactggaggccctggcagcaagctgtaaggacctgcgggagctgagagtgttcccatctgatccctttgtggccgagcctaacgtgagcctgaccgagcagggactggtgagcgtgtccggaggatgcccaaagctgcactccgtgctgtacttctgtagacagatgtctaatgccgccctgaccacaatcgcaaggaacaggcccaatttcacatgctttaggctgtgcatcatcgagcctcgcaccccagactatctgacactgggacctctggatgcaggatttggagcaatcgtggagcactgcaaggacctgagaaggctgtctgtgagcggcctgctgaccgatagggccttcgagtacatcggcacatatgccaagaagctggagatgctgagcctggcctttgccggcgactccgatctgggactgcaccacgtgctgagcggatgcgagaacctgcggaagctggagatcagagactgtcctttcggcgataaggccctgctggccaatgccgccaagctggagaccatgaggagcctgtggatgtcctcttgcatggtgtcctacggcgcctgtaagctgctgggccagaagatgccacgcctgaatgtggaagtgatcgacgagaggggaccacctgataccaggccagagtcctgtcccgtggagaagctgtacatctatcggtctgtggccggccctagattcgacatgccaggcttcgtgtggacaatggacgaggatagctccgccctgagactgtcc

SEQ ID NO: 103 - DNA, 1716 bp, Artificial Sequence
  GhAFB2 cDNA codon optimized
atgaactacttccctgacgaggtgctggagcacgtgttcgactttatcacctcccacaaggataggaattctgtgagcctggtgtgcaagagctggtacaagatcgagcggtgctcccggcagagagtgttcatcggcaactgctattccatctctcctgagaggctgatcgcccgctttccaggcctgaagtctctgacactgaagggcaagccacacttcgccgactttaatctggtgccccacgattggggcggcttcgtgtacccttggatcgaggccctggcaaagagcaggatcggactggaggagctgcggctgaagagaatggtggtgtctgacgagagcctggagctgctgagcaagtccttcgtgaacttcaagtccctggtgctggtgtcttgcgagggcttcaccacagacggactggcagcaatcgcagcaaactgtaggtttctgcgcgagctggatctgcaggagaatgaggtggacgatcaccggggccactggctgtcctgcttcccagagtcctgtacctctctgatcagcctgaactttgcctgcctgagaggagaagtgaatctgggcgccctggagcggctggtgtctagaagccctaacctgaagtctctgcggctgaatagagccgtgccactggatacactgcagaagctgctgatgagggcaccacagctggtggacctgggcatcggcagctacgtgcacgatccattctccgaggcctataacaagctgaagatcgccatccagcggtgtaagagcatcagatccctgtctggcttcctggaggtggcaccacactgcatgtccgccatctaccctatctgtggcaacctgacctttctgaatctgtcttatgcccccggcctgcacggcaataagctgatgaagctgatccagcactgcaggaagctgcagcgcctgtggatcctggactgtatcggcgataagggactgggagtggtggccctgacctgcaaggagctgcaggagctgagagtgttcccatccgacccctttgaggcaggaaacgcagcagtgacagaggagggactggtgctggtgtctgccggatgccctaagctgaacagcctgctgtacttctgtcagcagatgaccaatgccgccctgatcacagtggccaagaactgcccaaatttcatcaggtttcgcctgtgcatcctggacccaatcaagcccgatcctgtgaccaatcagcccctggacgagggctttggcgccatcgtgcagagctgtaagggcctgaagaggctgagcctgtccggcctgctgacagatcaggtgttcctgtacatcggcatgtatgccgagcagctggagatgctgtctatcgcctttgccgccgacagcgataagggcatgctgtacgtgctgaacggctgcaagaagctgaggaagctggagatcagggactgtcccttcggcgatgccgccctgctggaggacgtgggcaagtatgagaccatgcgcagcctgtggatgagctcctgcgaggtgacactgggcggctgtaagtccgtggccgagaagatgccttctctgaacgtggagatcatcgatgagtccgagcagatggagtttaatctggacgataagcagaaggtggacaagatgtacctgtatcggaccctggtgggccacagaaaggatgcccccgagtacgtgtggatcctg

SEQ ID NO: 104 - DNA, 1725 bp, Artificial Sequence
  NcAFB2 cDNA codon optimized
atgaactacttcccagacgaggtgaccgagcaggtgttcgactttgtgacaagccacaaggatcggaatgccatcagcctggtgtgcaagtcctggtacaagatcgagagatattcccggcagagagtgttcatcggcaactgctatgccatcaatcctgagaggctgctgcggagatttccatgtctgaagtccctgaccctgaagggcaagccacacttcgccgacttcaacctggtgccacacgagtggggaggcttcctgcagccttggatcgaggccctggcaaggtcccacgtgggactggaggagctgcggctgaagagaatggtggtgacagacgagtctctggagctgctgtcccgctctttcagcaactttaagtctctggtgctggtgagctgcgagggctttaccacagatggcctggccagcatcgccgccaactgtaggcacctgcgcgacctggatctgcaggagaatgagatcgacgatcaccgcggccagtggctgtcctgcttccctgacacctgtacatctctggtgaccctgaactttgcctgcctggagggcgagacaaatctggtggccctggagcggctggtggcaagatccccaaagctgaagtctctgaagctgaacagggcagtgccactggacgcactggcaagactggtggcatgtgcacctcagatcgtggagctgggagtgggaagctacgagaatgatcgggagtccgagcagtatctgaagctggaggccgccatcaagaagtgcacctccctgagatccctgtctggcttcctggaggcagcaccacacagcctgtccgcctttcaccctatctgtcacaacctgacatccctgaatctgtcttacgccgccgagatccacagctcccacctgatcaagctgatccagcactgcaacaagctgcagcggctgtggatcctggactctatcggcgataagggcctggaggtggtggccagcacctgtaaggagctgcaggagctgagagtgttcccttccgacctggtgggaggaggaaataccgcagtgacagaggatggactggtggcaatcagcgccggatgcccaaagctgcactccatcctgtatttttgtcagcagatgaccaacgccgccctgatcacagtggccaagaactgcccaaatttcatcaggtttcgcctgtgcatcctggagccaaataagccagaccacgtgaccagcaggccactggatgagggattcggagcaatcgtgagggcatgcaagcgcctgaggcgcctgtctctgagcggactgctgaccgaccaggtgttccactacatcggcaagtatgcccacgagctggagatgctgtctatcgcctttgccggcgacacagataagggcatgctgtacgtgctggacggctgtaagaagatgcggaagctggagatcagagacagcccctttggcgatgccgccctgctggcagacgtgagcaagtatgagaccatgaggagcctgtggatgtctagctgcgaggtgacactgggcggctgtaagcgcatcgcccagatcgccccttggctgaacgtggagatcatcaacgagaatgataacaataggatggagcagaatggacacgagggccgccaggaggtggacaagctgtacctgtataggaccgtggtgggaaccaggacagatgcaccacctttcgtgtggatcctg

SEQ ID NO: 105 - DNA, 1719 bp, Artificial Sequence
  MnAFB2 cDNA codon optimized
atgaactacttcccagatgaagtgatcgagcacgtgtttgattatgtgaccacacacaaggaccgcaatgccctgtctctggtgtgcaagagctggtaccggatcgagagactgtctaggctgcgcgtgttcatcggcaactgttatgccatcagccccgagagaaccgtggagaggtttcctggcctgcggtccctgacactgaagggcaagccacacttcgccgactttaatctggtgccacacgagtggggaggattcgtgtttccttggatcgaggccctggcacggagcagagtggccctggaggagctgaggctgaagcgcatggtggtgtctgatgagagcctggagctgctgtctagaagcttcgccaactttaagtccctggtgctggtgtcttgcgagggcttcaccacagacggactggcagcaatcgcagcaaattgtcggcacctgagagagctggatctgcaggagaacgagatcgaggacaataggggccagtggctgtcctgcttcgccgataactgtacctccctggtgtctctgaattttgcctgcctgaagggagagatcaacctggccgccctggagcgcctggtggcacggtctcctgatctgagggtgctgagggtgaatagggcagtgccactggacgcactgcagaagatcctgatgaaggcaccacagctggtggacctgggaaccggctcctacgtgacagattcccgctctgacgcctataacaagctgaagtctaccctgctgaagtgtcagagcatccggagcctgtccggcttcctggaggtggtgccttggtgcctgccagccttttaccccgtgtgcagcaacctgacatccctgaatctgtcttacgcccccggcatctatggcttcgagctgatcaagctgatcagacactgcgtgaagctgcagaggctgtggatcctggattgtatcggcgacaagggactgagcgtggtggcatccacctgcaaggagctgcaggagctgcgcgtgttcccaagcgatccatacggactgggacacgcagcagtgacagaggagggactggtggcaatcagcgtgggatgccctaagctgcactccctgctgtatttttgtcagcagatgaccaatgccgccctgatcacagtggccaagaactgcccaaatttcatcaggtttcgcctgtgcatcctggatccaaccaagcccgaccctgtgacatcccagccactggatgagggattcggagcaatcgtgcaggcctgtaagagcctgcggagactgtccctgaccggactgctgacagaccaggtgttcctgtacatcggcatgtatgccgagcagctggagatgctgtccgtggcctttgccggcgattctgacaagggcatgctgtacgtgctgcggggctgcaagaagctgagaaagctggagatcagggattgtccctttggcgacgtggccctgctgaccgacgtgggcaagtatgagacaatgaggtctctgtggatgagctcctgcgaggtgaccctgggagcctgtaagacagtggccaggaagatgcctagcctgaacgtggagatcatcaatgagcacgatcagaccgagttctgcctggacgatgacgagcagaaggtggagaagatgtacctgtataggaccacagtgggacctaggaccgacgcaccagagttcgtgtggaccctg

SEQ ID NO: 106 - protein, 9 aa, Artificial Sequence
  NLSweak (MycA1)
AAAKRVKLD

SEQ ID NO: 107 - protein, 9 aa, Artificial Sequence
  NLSstrong (Myc)
PAAKRVKLD

SEQ ID NO: 108 - protein, 7 aa, Simian virus 40
PKKKRKV

SEQ ID NO: 109 - DNA, 23 bp, Homo sapiens
accccacagtggggccactaggg

SEQ ID NO: 110 - DNA, 23 bp, Homo sapiens
gtcaccaatcctgtccctagtgg

SEQ ID NO: 111 - protein, 5 aa, Artificial Sequence
  Linker GGSGG
GGSGG

SEQ ID NO: 112 - protein, 8 aa, Artificial Sequence
  NES peptide sequence
LDLASLIL

SEQ ID NO: 113 - protein, 14 aa, Artificial Sequence
  NES21
IDELLKELADLNLD

SEQ ID NO: 114 - protein, 102 aa, Arabidopsis thaliana
KRGFSETVDLKLNLNNEPANKEGSTTHDVVTFDSKEKSACPKDPAKPPAKAQVVGWPPVRSYRKNVMVSCQKSSGGPEAAAFVKVSMDGAPYLRKIDLRMYK

SEQ ID NO: 115 - protein, 112 aa, Arabidopsis thaliana
KRGFSETVDLMLNLQSNKEGSVDLKNVSAVPKEKTTLKDPSKPPAKAQVVGWPPVRNYRKNMMTQQKTSSGAEEASSEKAGNFGGGAAGAGLVKVSMDGAPYLRKVDLKMYK

SEQ ID NO: 116 - protein, 103 aa, Arabidopsis thaliana
KRGFSETVDLKLNLQSNKQGHVDLNTNGAPKEKTFLKDPSKPPAKAQVVGWPPVRNYRKNVMANQKSGEAEEAMSSGGGTVAFVKVSMDGAPYLRKVDLKMYT

SEQ ID NO: 117 - protein, 78 aa, Arabidopsis thaliana
KRVLSTDTEKEIESSSRKTETSPPRKAQIVGWPPVRSYRKNNIQSKKNESEHEGQGIYVKVSMDGAPYLRKIDLSCYK

SEQ ID NO: 118 - DNA, 32 bp, Homo sapiens
aagtcaccatggcacagcaagctgccgataag

SEQ ID NO: 119 - DNA, 32 bp, Homo sapiens; identical to SEQ ID NO: 118

SEQ ID NO: 120 - DNA, 30 bp, Homo sapiens
ccaacctgccggccatggagaccccgtccc

SEQ ID NO: 121 - DNA, 29 bp, Homo sapiens
ccgccatggcgactgcgacccccgtgccg

SEQ ID NO: 122 - DNA, 29 bp, Homo sapiens
ccatacatctactaatgctcttctggctt

SEQ ID NO: 123 - DNA, 29 bp, Homo sapiens
ttctaaatttctagccctctcgcagggca

SEQ ID NO: 124 - DNA, 29 bp, Homo sapiens
gtgaatttattggagcatgaccacggagg

SEQ ID NO: 125 - DNA, 32 bp, Homo sapiens
tgattcccaagtgtgagtcgccccagatcacc

SEQ ID NO: 126 - DNA, 48 bp, Homo sapiens
gagtaaacttttctagctgcccctttctgtaatagtgaaagttggtat

SEQ ID NO: 127 - DNA, 28 bp, Homo sapiens
catctccaatatggtatggcggcccttc

SEQ ID NO: 128 - DNA, 29 bp, Homo sapiens
acctgctctagttcctgaagaaaaggggc

SEQ ID NO: 129 - DNA, 29 bp, Homo sapiens
ccctcagcaactggagaaatgatttttcc

(19) United States
(12) Patent Application Publication
(10) Pub. No.: US 2022/0041665 A1
(43) Pub. Date: Feb. 10, 2022

(54) POLYNUCLEOTIDE AND USES THEREOF

(71) Applicant: Helsingin yliopisto, Helsingin yliopisto (FI)

(72) Inventors: Shiqian LI, Helsinki (FI); Elina IKONEN, Helsinki (FI)

(73) Assignee: Helsingin yliopisto, Helsingin yliopisto (FI)

(21) Appl. No.: 17/593,859

(22) PCT Filed: Mar. 26, 2020
(86) PCT No.: PCT/FI2020/050196

§ 371 Date: Sep. 27, 2021

(30) Foreign Application Priority Data
Mar. 27, 2019 (FI) 20195239

(51) Int. Cl. C07K 14/415; C12N 15/63

(52) U.S. Cl. CPC C07K 14/415; C07K 2319/95; C12N 15/63

(57) ABSTRACT

A polynucleotide encoding a degradation signal peptide is disclosed. The polynucleotide may comprise a nucleotide sequence encoding a degradation signal peptide, wherein the degradation signal peptide has an amino acid sequence comprising a sequence that is at least 75% identical to a sequence corresponding to amino acid residues 84-98 of SEQ ID NO: 1 (AtIAA7), 66-80 of SEQ ID NO: 2 (AtIAA3), 84-98 of SEQ ID NO: 3 (AtIAA17), 78-92 of SEQ ID NO: 4 (AtIAA14), 55-69 of SEQ ID NO: 5 (AtIAA5), or 167-181 of SEQ ID NO: 6 (AtIAA8), or a degradation signal peptide functionally and/or structurally equivalent thereto.

### TECHNICAL FIELD

The present disclosure relates to a polynucleotide, a polypeptide or protein, an expression cassette, a vector, a system, a kit, a host cell, a transgenic organism, a method for at least partially depleting a target polypeptide or protein in a host cell, a method for producing the host cell, and use thereof.

### BACKGROUND

Targeted protein degradation, i.e. depletion, of endogenous polypeptides and proteins using small molecules as inducers, may be desirable for various purposes, for example the study of the function of individual proteins or assessment of drug targets. The auxin-inducible degron (AID) technique may be used to control targeted protein degradation with the small molecule auxin or an auxin analogue.

However, in many cases the complete or partial basal degradation of a protein, i.e. constitutive depletion, in the absence of an inducer such as auxin or an auxin analogue, may result in adverse consequences. Some proteins are also essential, so that even a partial basal degradation of such a protein may result in severe consequences, for example cell death. The ability to rapidly and efficiently induce the degradation of proteins may therefore be very useful.

In some systems, the inducible degradation may be inefficient. Some AID systems may be sensitive to higher temperatures, for example to a temperature of 37° C. typical for mammalian cells. Furthermore, certain types of proteins may be more challenging to degrade inducibly than others. The system used for the degradation and/or the inducer thereof should preferably also not cause excessive side effects.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A polynucleotide encoding a degradation signal peptide is disclosed. The polynucleotide may comprise a nucleotide sequence encoding a degradation signal peptide, wherein the degradation signal peptide has an amino acid sequence comprising a sequence that is at least 75% identical to a sequence corresponding to amino acid residues

84-98 of SEQ ID NO: 1 (AtIAA7),

66-80 of SEQ ID NO: 2 (AtIAA3),

84-98 of SEQ ID NO: 3 (AtIAA17),

78-92 of SEQ ID NO: 4 (AtIAA14),

55-69 of SEQ ID NO: 5 (AtIAA5), or

167-181 of SEQ ID NO: 6 (AtIAA8),

or a degradation signal peptide functionally and/or structurally equivalent thereto.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 is a schematic representation of the principle of rapid protein degradation with AID;

FIG. 2A shows the scheme for screening auxin perceptive proteins and degron tags. Target protein levels in cells are analyzed by FACS of GFP at 0 h IAA for basal degradation, at 1 h IAA for early degradation efficiency and at 16 h IAA for final degradation efficiency;

FIG. 2B illustrates mean GFP intensity analyzed as shown in FIG. 2A. miniAID as the degron (n=4, OsTIR1 and AtAFB2; Ctrl (control), no auxin perceptive protein); or AtAFB2 as the auxin perceptive protein (n=2, miniAID; n=4, miniIAA7; Ctrl, Seipin-mEGFP without degron);

FIGS. 2C and 2D show FACS plots showing overlays of GFP histograms at 0 h (black line), 1 h (grey line) and 16 h (light grey line) IAA in cells expressing the indicated constructs. Ctrl: seipin-mEGFP without degron. *: KR dipeptide in domain II; black dotted line and grey dotted line: drawings for comparison of GFP peaks. The plots show that miniIAA7 and KR-miniIAA7 had the best reduction of GFP at 1 h IAA in AtAFB2 expressing cells. KR domain as a potential nuclear localization signal is unfavourable to be included in the tag;

FIG. 2E shows mean GFP intensities in FIGS. 2C and 2D (n=2-4). N.D. not determined;

FIG. 3A illustrates the scheme for establishing cell lines with AtAFB2-miniIAA7 system to deplete endogenous proteins. Safe harbor locus with control (Ctrl) or OsTIR1 were used for comparison;

FIG. 3B shows mean GFP intensity analyzed by FACS in A431 cells with miniIAA7-tagged DHC1 (n=4) and A549 cells with miniIAA7-tagged EGFR (n=2). The cells also expressed indicated auxin-perceptive proteins or Ctrl. N.A. not available due to inability to establish the cell line;

FIG. 3C shows time-lapse images of A431-DHC1 cells with or without cell division after mitotic cell rounding. Open arrowhead: cells before mitosis; filled arrowhead: cells undergoing mitotic rounding; arrow: cells after cell division;

FIG. 3D shows analysis of the fraction of cell division after mitotic rounding (n=12 fields, 124-185 cells/condition);

FIG. 3E shows Alexa647 EGF uptake analyzed by FACS in A549-EGFR cells (n=2);

FIG. 4A illustrates the comparison of miniIAA7-mEGFP and mEGFP-miniIAA7 as N-terminal tags in depleting endogenous SEC61β. Scheme showing establishment of A431 cell lines using miniIAA7-mEGFP (1.) or mEGFP-miniIAA7 (2.) to tag endogenous SEC61B N-terminally;

FIG. 4B shows relative GFP intensities analyzed by FACS in homozygously tagged Ctrl cells at 0 h IAA (n=3);

FIG. 4C shows live cell airyscan images showing similar endoplasmic reticulum localization of tagged proteins;

FIG. 4D shows mean GFP intensities analyzed by FACS in cells with indicated endogenously miniIAA7-tagged Sec61B and auxin-perceptive proteins or Ctrl (n=3);

FIG. 5A illustrates depletion of endogenous transmembrane, cytoplasmic and nuclear proteins using AtAFB2-miniIAA7 system. Scheme for N- or C-terminal tagging of endogenous target locus with miniIAA7-mEGFP;

FIG. 5B is an illustration of subcellular localization of target proteins. Numbers represent transmembrane segments; ER: endoplasmic reticulum; LD: lipid droplet; LE: late endosome; N: nucleus; PM: plasma membrane; PX: peroxisome;

FIG. 5C shows relative target protein levels analyzed by FACS of mean GFP intensity in Ctrl cell lines at 0 h IAA. All targets were tagged homozygously in A431 cells (n=3-5);

FIG. 5D shows mean GFP intensity analyzed by FACS in cells with endogenously miniIAA7-tagged targets and indicated auxin-perceptive proteins or Ctrl (n=3-5);

FIG. 5E is a scheme for increasing AtAFB2 nuclear localization (upper panel) and images of AtAFB2-mCherry without NLS, with weak or strong NLS in A431 cells;

FIG. 5F shows mean GFP intensity analyzed by FACS in cells with endogenously miniIAA7-tagged nuclear proteins and different AtAFB2-mCherry constructs (n=3);

FIGS. 5G-L show loss-of-function phenotypes in cells with proteins targeted for degradation using the AtAFB2-miniIAA7 system. FIG. 5G: Auxin-inducible reduction in glucose uptake in cells with tagged Glut1 (n=9); FIG. 5H, phalloidin staining showing auxin-inducible changes in F-actin structures in cells with tagged NMIIa. Maximum intensity projections of deconvolved widefield images are shown; FIG. 5I, widefield images of filipin stained cells showing auxin-inducible perinuclear cholesterol accumulation in cells with tagged NPC1; FIG. 5J, LD540 staining of lipid droplets showing auxin-inducible changes in LDs in cells with tagged seipin. Right: quantification of fraction of tiny LDs<0.05 um²/cell. Data represent mean±SEM (n>200 cells per condition); FIG. 5K, auxin-inducible reduction in cellular cholesterol content in cells with tagged LBR (n=4); FIG. 5L, auxin-inducible reduction in peroxisomal membrane proteins in cells with tagged PEX3. Top left: western blot analysis of endogenous PMP70 levels. Top right and bottom: Images and quantification of overexpressed PMP22-mCardinal fluorescence;

FIGS. 6A and 6B show characterization of AtTIR1, AtAFB2 and miniIAA7 through atomistic molecular dynamics simulations. FIG. 6A, schematic representation, and FIG. 6B, table characterizing the amino acid residues of IAA binding pocket involved in IAA binding in AtTIR1 and AtAFB2 by simulations (n=5). AtTIR1 backbone is shown in the background as transparent. IAA is depicted in van der Waals representation. Residues defining IAA binding pockets are illustrated in blue/licorice representation, with AtTIR1 residues in darker blue and AtAFB2 residues in lighter blue. Residue numbers refer to those of AtTIR1. Residues in larger font represent ones involved in interaction with IAA in the simulations and in the crystal structure (PDB ID: 2P1P), red residue numbers represent ones involved in IAA interaction in AtTIR1 but not in AtAFB2. Degrons miniIAA7-V1 and -V2 are schematically illustrated in FIG. 6F;

FIGS. 6C and 6D are representative snapshots highlighting miniIAA7-V1 and -V2 degrons in the indicated complexes at the end of 1 μs simulations (n=5). Magenta: N-terminal KR dipeptide; brown: aa. 95-104; pink: C-terminal extension after 5104;

FIG. 6E shows secondary structure plots for each amino acid of miniIAA7-V1 and miniIAA7-V2. The values describing the probability of observing different secondary structures (alpha helix, coil, beta sheet, turn) have been averaged over the simulation period and replicas (n=5); and

FIG. 6F shows FACS analysis of mean GFP intensities at 0 h (black), 1 h (grey) and 16 h (no fill) IAA in cells expressing AtAFB2 and seipin-mEGFP with the indicated truncated AtIAA7 degron insertions (n=2-3). Ctrl: seipin-mEGFP without degron. *: KR dipeptide in domain II. The results show that a minimal degron with similar performance to miniIAA7 located in aa.82-101.

### DETAILED DESCRIPTION

A polynucleotide comprising a nucleotide sequence encoding a degradation signal peptide is disclosed.

With the polynucleotide encoding the degradation signal peptide, nucleic acid cassette, vector, system and methods according to one or more embodiments described in this specification, and by using an inducer such as auxin or an auxin analogue, it may be possible to deplete target polypeptides or proteins in mammalian and other host cells rapidly and highly efficiently, i.e. to target them to rapid degradation. Relatively high depletion efficiencies after e.g. only 1 hour of adding auxin or an auxin analogue may be achieved.

In some embodiments, half-times of minutes may be achieved, and the targeted proteins may be depleted to very low, even nearly background levels. Therefore, the depletion may lead to clear phenotypes in the host cell or organism. The inducer, i.e. auxin, such as the commonly used inducer indole-3-acetic acid (IAA), or auxin analogue, may be relatively safe, economical, small in size, may be applied to a culture medium and may be reversible by washing. Few or no growth defects are typically observed, and little or no differential gene activity are typically detected in cultured cells.

It may also be possible to avoid at least partial basal degradation of the target polypeptide or protein, i.e. constitutive depletion, or reduce the extent of the at least partial basal degradation. However, the extent of basal degradation may be specific to content and target polypeptide or protein. At least partial basal degradation may be challenging to completely avoid, so there may in most cases be at least some basal degradation.

In the context of this specification, the term “basal degradation” may be understood as referring to constitutive depletion, i.e. to degradation of the target polypeptide or protein that may occur in the absence of an inducer. Each polypeptide or protein may have a an intrinsic degradation rate, i.e. protein turnover, that is characteristic of the polypeptide or protein and is due to the cellular machinery, e.g. the proteolytic machinery, and its normal biochemical functioning. However, the presence of a functional auxin perceptive protein, polynucleotide, polypeptide or protein, fusion protein, expression cassette, vector or system according to one or more embodiments described in this specification may (but does not necessarily) increase the extent of the degradation. Thus “basal degradation” and/or the extent thereof may, at least in some embodiments, be understood as referring to constitutive depletion, i.e. to degradation of the target polypeptide or protein that may occur in the absence of an inducer but in the presence of a functional auxin perceptive protein, polynucleotide, polypeptide or protein, fusion protein, expression cassette, vector or system according to one or more embodiments described in this specification, for example in an otherwise comparable host cell. In other words, the term “basal degradation” may be understood as referring to the degradation of a target polypeptide or protein in the presence of a functional inducible system for depletion of the target polypeptide or protein in an uninduced state, i.e. in the absence of an inducer. The basal degradation may thus be understood as accelerated degradation (as compared to the intrinsic degradation) caused by the uninduced interaction of the degradation signal peptide with the functional auxin perceptive protein. In other words, the basal degradation or the extent thereof may be calculated, for example, by measuring the proportion of the amount of the degraded target polypeptide or protein relative to the amount of the target polypeptide or protein in the absence of a functional auxin perceptive protein in the host cell(s), tissue or organism.

In an embodiment, basal degradation of the target polypeptide or protein is at most 50%, or at most 40%, or at most 30%, or at most 20%, or at most 15%, in the absence of an inducer but optionally in the presence of a functional auxin perceptive protein, the polynucleotide, the polypeptide, the expression cassette, the vector, and/or of the system according to one or more embodiments described in this specification. In other words, the target polypeptide or protein is present at a level that is at most 50%, or at most 40%, or at most 30%, or at most 20%, or at most 15%, lower in the absence of an inducer but optionally in the presence of a functional auxin perceptive protein, the polynucleotide, the polypeptide, the expression cassette, the vector, and/or of the system according to one or more embodiments described in this specification, in a host cell, for example in a host cell according to one or more embodiments described in this specification.

Furthermore, it may be possible to deplete target polypeptides or proteins that may be otherwise challenging to deplete, for example membrane proteins and other large proteins, or proteins the basal degradation of which is highly deleterious to cells.

The depletion is not particularly sensitive to higher temperatures, for example to a temperature of about 37° C. typical for maintaining mammalian cells.

The degradation signal peptide may be relatively short and therefore may minimize any interference to the function of the target polypeptide or protein, or a moiety capable of associating with the target polypeptide or protein, to which it is fused.

Furthermore, degradation signal peptides which do not contain the PB1 domain of AtIAAs or amino acid sequences thereof appear to be highly efficient.

The degradation signal peptides appear to be highly efficient, when used together with AtAFB2 as the functional auxin perceptive protein.

In the context of this specification, the terms “degradation signal peptide”, “destabilizing domain”, “auxin-inducible destabilizing domain”, “degron” or “degron tag” may refer to a peptide, a polypeptide or a protein that is capable of targeting it and any protein or polypeptide fused to it or otherwise associated with it for degradation by the proteasome. These terms may be used interchangeably.

Generally, the term “peptide” may be understood as referring to a peptide chain of about 2 to 50 amino acid residues, and the term “protein” as referring to a peptide chain of more than 50 amino acid residues. The term “polypeptide” may commonly be used to refer to a peptide chain of any length, or e.g. to a peptide chain of about 10 to 100 amino acid residues. However, the term “peptide” may also be used to denote a peptide chain of at least 2 amino acid residues, not limited to any particular length. Therefore, as a skilled person is aware, there may be a great deal of overlap between these terms, and they may be used interchangeably at least to some extent. The terms “peptide”, “polypeptide” and “protein” are therefore not intended to define peptide chains of any particular length, unless otherwise indicated. The phrase “polypeptide or protein” is intended to cover peptide chains of any possible length.

In the context of this specification, the term “polynucleotide” may be understood as referring to a chain of nucleotides, such as DNA and/or RNA, of any length. The polynucleotide may be, for example, DNA, RNA, cDNA, mRNA, or any combination thereof. The polynucleotide may be, for example, linear, circular or branched. The nucleotides of the polynucleotide may be naturally occurring and/or synthetic nucleotides, for example nucleotide analogues. The polynucleotide may also comprise one or more modifications, for example a label.

In the context of this specification, the term “nucleotide sequence encoding a degradation signal peptide” may be understood as referring both to the nucleotide (i.e. a polynucleotide or a part thereof) as well as to its amino acid sequence.

The terms “depleting” or “depletion” may be understood as referring to a reduction in the amount and/or concentration of a target polypeptide or protein, for example in a host cell or transgenic organism. The depletion may be achieved by targeted, inducible degradation of the target polypeptide or protein, for example using an AID system. The depletion may thus be induced by using an inducer. In the context of this specification, the terms “inducible degradation” or “inducibly degrade” may thus be understood as depletion, i.e. degradation of the target polypeptide or protein that may be caused by the presence and/or addition of an inducer, e.g. an auxin or an auxin analogue. Various examples of depletion, i.e. inducible degradation are described in this specification. The depletion may further require the presence of a functional auxin perceptive protein, polynucleotide, polypeptide or protein, fusion protein, expression cassette, vector or system according to one or more embodiments described in this specification.

The terms “depleting” or “depletion” may be understood as referring to partial or complete depletion. 100%, i.e. complete, depletion of a protein may be challenging to achieve, so typically depletion efficiencies lower than 100% or 1, i.e. partial depletion, are achieved. Thus the word “depleting” or “depletion” may not be understood as referring to complete depletion, unless specifically mentioned as such. The depletion efficiency may be, for example, at least 50%, or at least 60%, or at least 70%, or at least 80%, or at least 90%, at a given time period, for example within 1 hour. The depletion efficiency may be calculated, for example, by measuring the proportion of the amount of the depleted target polypeptide or protein relative to the amount of the target polypeptide or protein at time point 0 (i.e. immediately before the addition of the inducer), or relative to the amount of the target polypeptide or protein in the absence of a functional auxin perceptive protein in the host cell(s), tissue or organism. In other words, the depletion efficiency may be calculated relative to the amount of the target polypeptide or protein in host cell(s), tissue(s) or organism which does not express or contain a functional auxin perceptive protein. The amounts or levels of the target polypeptide or protein may further be calculated and/or normalized relative to the amount or level of the target polypeptide or protein in control cells or organism without a functional auxin perceptive protein at time point 0 (i.e. immediately before the addition of the inducer). This may also allow measuring the extent of possible basal degradation.

For instance, in the present Examples, target protein levels (amounts) have been measured without the presence of a functional auxin perceptive protein at 0 h IAA to normalize all the data. This also allows for measuring the extent of basal degradation. As shown in the Examples, the depletion efficiency may be calculated as the normalized level of the target polypeptide or protein at an indicated IAA treatment time point compared to a control without a functional auxin perceptive protein at 0h IAA (or, in other embodiments, another inducer). The depletion may include basal degradation and/or inducible degradation. For example, if a target polypeptide or protein is present at 90% level at 0 h IAA as compared to a control not expressing a functional auxin perceptive protein, the basal depletion efficiency is 10%. After 1h IAA, the target polypeptide or protein may be present at 5% level as compared to a control not expressing a functional auxin perceptive protein. Then the inducible degradation is 85%, and the total depletion efficiency is 95% (10% basal+85% inducible).

In the context of this specification, the term “target polypeptide”, “target protein”, or “target gene” may be understood as referring to the polypeptide, protein or gene of interest for depletion. The target gene may encode the target polypeptide or protein.

In the context of this specification, the term “inducer” may be understood as referring to an auxin, an auxin analogue, or any other agent capable of binding to a functional auxin perceptive protein, thereby inducing at least a partial depletion (induced degradation) of a target polypeptide or protein. Upon the binding, the functional auxin perceptive protein may bind to the degradation signal peptide, thereby inducing the art least partial depletion of the target polypeptide or protein.

In the context of this specification, the term “auxin” may be understood as referring to any compound belonging to the auxin class of plant hormones. The term may encompass auxins occurring naturally in plants, including indole-3-acetic acid (IAA), 4-chloroindole-3-acetic acid (4-CI-IAA), 2-phenylacetic acid (PAA), indole-3-butyric acid (IBA), and indole-3-propionic acid (IPA), as well as synthetic auxins, including 2,4-dichlorophenoxyacetic acid (2,4-D), α-naphthalene acetic acid (α-NAA), 2-methoxy-3,6-dichlorobenzoic acid (dicamba), 4-amino-3,5,6-trichloropicolinic acid (tordon or picloram), and 2,4,5-trichlorophenoxyacetic acid (2,4,5-T). The term “auxin analogue” may refer to a derivative of an auxin. For example, the auxin analogue may comprise a derivative of IAA, such as those compounds having a substituted moiety (not H) on the 4-position of the indole ring of IAA. Examples include e.g. 4-methylindole-3-acetic acid (4-Me-IAA), 4-chloroindole-3-acetic acid (4-Cl-IAA), or cvxIAA (5-(3-methoxyphenyl)indole-3-acetic acid. Other auxins and/or auxin analogues may also be contemplated, found in nature or synthesized. The auxin and/or auxin analogue may be capable of binding to an auxin perceptive F-box protein, such as TIR1 and/or AFB2 (e.g. OsTIR1, AtAFB2 or other auxin perceptive F-box proteins described in this specification), or a derivative thereof, such as AtTIR1 F79G mutant or an F79G mutant of any other TIR1 protein. cvxAA and the AtTIR1 F79G mutant have been described e.g. in Uchida et al., Nature Chemical Biology 2018, 14, 299-305.

In the context of this specification and in the context of any product, method or use disclosed herein, the terms “host cell” and/or “host genome” may be understood as referring to a host cell or host genome of any genus or species. The host cell may be an animal cell or a fungal cell. The host genome may be an animal genome or a fungal genome. The host cell may be a eukaryotic cell. The host genome may be a eukaryotic genome. The host cell may be a mammalian cell, for example a human, murine, bovine, ovine, porcine, feline, canine, equine, or primate cell; a nematode cell; a fish cell; or an insect cell. The host genome may be the genome of any one of the host cells and/or transgenic organisms described in this specification. The host genome may be a mammalian genome, for example a human, murine, bovine, ovine, porcine, feline, canine, equine, or primate genome; a nematode genome; a fish genome; or an insect genome. In an embodiment, the host cell is a host cell or eukaryotic cell other than a plant cell. In an embodiment, the host genome is a genome or eukaryotic genome other than a plant genome.

The degradation signal peptide may have an amino acid sequence comprising a sequence that is at least 75% identical, or at least 80% identical, or at least 85% identical, or at least 90%, or at least 95% identical, or 100% identical, to a sequence corresponding to amino acid residues at positions

84-98 of SEQ ID NO: 1 (AtIAA7),

66-80 of SEQ ID NO: 2 (AtIAA3),

84-98 of SEQ ID NO: 3 (AtIAA17),

78-92 of SEQ ID NO: 4 (AtIAA14),

55-69 of SEQ ID NO: 5 (AtIAA5), or

167-181 of SEQ ID NO: 6 (AtIAA8);

or it may be a degradation signal peptide functionally and/or structurally equivalent thereto.

Such a sequence, and various embodiments thereof described below, may be considered a core sequence. The core sequence may provide the functionality of the degradation signal peptide. Other parts and sequences of the polynucleotide may or may not affect the functionality, efficiency etc. of the degradation signal peptide the sequence encodes.

To determine the extent of identity of two sequences, methods of alignment are well known in the art. Thus, the determination of percent identity between any two sequences can be accomplished using a mathematical algorithm such as the algorithm described by Lipman and Pearson (Science 1985, 227(4693), 1435-1441). For example, the ClustalW or ClustalΩ software may be used for the alignment. The sequences set forth in this specification are provided as non-limiting examples. A person skilled in the art will appreciate that other sequences, e.g. paralogs or orthologs, and providing the same activity or functionality may be found in other species or genetic backgrounds or produced artificially; these sequences may be considered substantially similar, i.e. representing functional and structural equivalents. The percentage identity may be relative to the full length of the reference sequence to which the sequence in question is compared, or based on a partial alignment.

The term “functionally and/or structurally equivalent thereto” may, in the context of this specification, be understood as referring to a degradation signal peptide that does not necessarily have the same sequence or sequence identity defined in one of more embodiments described in this specification, but which is capable of performing the same function in substantially the same way. The functional and/or structural equivalent may have substantially the same secondary structure, fully or at least partially. However, it does not necessarily have exactly the same secondary structure. The structural equivalence of a degradation signal peptide may be assessed e.g. by molecular dynamics simulations as described in the Examples of the present specification. For example, the C-terminal part of the degradation signal peptide may have a flexible coil structure, e.g. when interacting with a functional auxin perceptive protein capable of binding the degradation signal peptide in the presence of auxin or an auxin analogue (e.g. AtAFB2). This may be opposed to e.g. an alpha-helical structure, which certain IAA-derived degradation signal peptides, such as IAA7 extending to or beyond AA residue 124 of SEQ ID NO: 1, may adopt. The functional equivalence may be assessed by measuring the functioning, e.g. as described in the Examples.

The degradation signal peptide may have an amino acid sequence comprising a sequence that is at least 75% identical, or at least 80% identical, or at least 85% identical, or at least 90%, or at least 95% identical, or 100% identical, to a sequence corresponding to amino acid residues

84-99 of SEQ ID NO: 1 (AtIAA7),

66-81 of SEQ ID NO: 2 (AtIAA3),

84-99 of SEQ ID NO: 3 (AtIAA17),

78-93 of SEQ ID NO: 4 (AtIAA14),

55-70 of SEQ ID NO: 5 (AtIAA5), or

167-182 of SEQ ID NO: 6 (AtIAA8);

or it may be a degradation signal peptide functionally and/or structurally equivalent thereto.

The degradation signal peptide may have an amino acid sequence comprising a sequence that is at least 75% identical, or at least 80% identical, or at least 85% identical, or at least 90%, or at least 95% identical, or 100% identical, to a sequence corresponding to amino acid residues

84-100 of SEQ ID NO: 1 (AtIAA7),

66-82 of SEQ ID NO: 2 (AtIAA3),

84-100 of SEQ ID NO: 3 (AtIAA17),

78-94 of SEQ ID NO: 4 (AtIAA14),

55-71 of SEQ ID NO: 5 (AtIAA5), or

167-183 of SEQ ID NO: 6 (AtIAA8);

or it may be a degradation signal peptide functionally and/or structurally equivalent thereto.

The degradation signal peptide may have an amino acid sequence comprising a sequence that is at least 75% identical, or at least 80% identical, or at least 85% identical, or at least 90%, or at least 95% identical, or 100% identical, to a sequence corresponding to amino acid residues

84-101 of SEQ ID NO: 1 (AtIAA7),

66-83 of SEQ ID NO: 2 (AtIAA3),

84-101 of SEQ ID NO: 3 (AtIAA17),

78-95 of SEQ ID NO: 4 (AtIAA14),

55-72 of SEQ ID NO: 5 (AtIAA5), or

167-184 of SEQ ID NO: 6 (AtIAA8);

or it may be a degradation signal peptide functionally and/or structurally equivalent thereto.

The degradation signal peptide may comprise or consist of a sequence represented by formula I

X₁X₂VGWPPX₃X₄X₅X₆X₇X₈ Formula I

wherein

X₁ is Q or absent;

X₂ is absent, V, I, A, or L;

X₃ is V, I, L, G, or A;

X₄ is R, C, or K;

X₅ is N or S;

X₆ is Y, F, or W;

X₇ is R or K; and

X₈ is K or R.

The sequence represented by formula I may thus form a subsequence of the degradation signal peptide and/or the core sequence. The degradation signal peptide may further comprise one or more additional (sub)sequences preceding or following the sequence represented by formula I. The one or more additional subsequences may immediately precede and/or immediately follow the sequence represented by formula I. Examples of such additional subsequences are described below in this specification.

The (sub)sequence represented by formula I may be selected from the following (i.e. the degradation signal peptide may comprise or consist of a sequence selected from the following, or an amino acid sequence comprising a sequence that is at least 75% identical, or at least 80% identical, or at least 85% identical, or at least 90%, or at least 95% identical, or 100% identical to the following, or it may be a degradation signal peptide functionally and/or structurally equivalent thereto):

| (SEQ ID NO: 7) | |
|---|---|
| | QVVGWPPVRNYRK, |
| (SEQ ID NO: 8) | |
| | QVVGWPPVRSYRK, |
| (SEQ ID NO: 9) | |
| | QIVGWPPVRSYRK, |
| (SEQ ID NO: 10) | |
| | QIVGWPPIRSYRK, |
| (SEQ ID NO: 11) | |
| | QVVGWPPIRSYRK, |
| (SEQ ID NO: 12) | |
| | QVVGWPPIRSFRK, |
| (SEQ ID NO: 13) | |
| | QVVGWPPVCSYRR, |
| (SEQ ID NO: 14) | |
| | QAVGWPPVCSYRR, |
| | and |
| (SEQ ID NO: 15) | |
| | QVVGWPPVRSYRR. |

The (sub)sequence represented by formula I may be followed by a (sub)sequence represented by formula II

X₉X₁₀X₁₁X₁₂X₁₃X₁₄X₁₅X₁₆X₁₇X₁₈ Formula II

wherein

X₉ is N, S, T, K, or R;

X₁₀ is M, I, V, N, S, T, or L;

X₁₁ is M, I, L, V, S, or T;

X₁₂ is T, A, V, G, Q, H, S, L, F, or I;

X₁₃ is absent, Q, N, H, T, S, A, E, P, I, or L;

X₁₄ is absent, Q, P, C, S, Y, K, N, R, or T;

X₁₅ is absent, K, Q, T, P, S, N, or R;

X₁₆ is absent, S, N, T, K, P, or A; and

X₁₇ is absent, S, G, A, P, E, T, N, K, or R;

X₁₈ is absent, S, E, T, G, or N.

The (sub)sequence represented by formula I may be immediately followed by the (sub)sequence represented by formula II, or they may be linked e.g. via a linker. For example, the linker may be a linker of at least one amino acid residue, or 1-5, 2, 3, 4, or 5, or 1-3 amino acid residues, or 1 amino acid residue.

The degradation signal peptide may comprise or consist of a sequence represented by formula I

X₁X₂VGWPPX₂X₄X₅X₆X₇X₈ Formula I

wherein

X₁ is Q or absent;

X₂ is absent, V, I, A, or L;

X₃ is V, I, L, G, or A;

X₄ is R, C, or K;

X₅ is N or S;

X₆ is Y, F, or W;

X₇ is R or K; and

X₈ is K or R;

optionally followed by a sequence represented by formula II

X₉X₁₀X₁₁X₁₂X₁₃X₁₄X₁₅X₁₆X₁₇X₁₈ Formula II

wherein

X₉ is N, S, T, K, or R;

X₁₀ is M, I, V, N, S, T, or L;

X₁₁ is M, I, L, V, S, or T;

X₁₂ is T, A, V, G, Q, H, S, L, F, or I;

X₁₃ is absent, Q, N, H, T, S, A, E, P, I, or L;

X₁₄ is absent, Q, P, C, S, Y, K, N, R, or T;

X₁₅ is absent, K, Q, T, P, S, N, or R;

X₁₆ is absent, S, N, T, K, P, or A; and

X₁₇ is absent, S, G, A, P, E, T, N, K, or R;

X₁₈ is absent, S, E, T, G, or N;

or it may be a degradation signal peptide functionally and/or structurally equivalent thereto. In this embodiment, the (sub)sequence represented by formula I may be immediately followed by the (sub)sequence represented by formula II, or they may be linked e.g. via a linker. For example, the linker may be a linker of at least one amino acid residue, or 1-5, 2, 3, 4, or 5, or 1-3 amino acid residues, or 1 amino acid residue.

The (sub)sequence represented by formula II may, in some embodiments, be selected from the following:

| (SEQ ID NO: 16) | |
|---|---|
| | NMMT, |
| (SEQ ID NO: 17) | |
| | NIMT, |
| (SEQ ID NO: 18) | |
| | NIIT, |
| (SEQ ID NO: 19) | |
| | NVMA, |
| (SEQ ID NO: 20) | |
| | NIMA, |
| (SEQ ID NO: 21) | |
| | SVMA, |
| (SEQ ID NO: 22) | |
| | TVMA, |
| (SEQ ID NO: 23) | |
| | NVMV, |
| (SEQ ID NO: 24) | |
| | NMMV, |
| (SEQ ID NO: 25) | |
| | NVMG, |
| (SEQ ID NO: 26) | |
| | NVLV, |
| (SEQ ID NO: 27) | |
| | NNIQ, |
| (SEQ ID NO: 28) | |
| | NNVQ, |
| (SEQ ID NO: 29) | |
| | NNIH, |
| (SEQ ID NO: 30) | |
| | NTMA, |
| (SEQ ID NO: 31) | |
| | NTMS, |
| (SEQ ID NO: 32) | |
| | KNSL, |
| (SEQ ID NO: 33) | |
| | KNSF. |

The (sub)sequence represented by formula II may, in some embodiments, be selected from the following:

| (SEQ ID NO: 34) | |
|---|---|
| | NMMTQQK, |
| (SEQ ID NO: 35) | |
| | NIMTQQK, |
| (SEQ ID NO: 36) | |
| | NIMTNQK, |
| (SEQ ID NO: 37) | |
| | NIITQQK, |
| (SEQ ID NO: 38) | |
| | NVMANQK, |
| (SEQ ID NO: 39) | |
| | NIMANQK, |
| (SEQ ID NO: 40) | |
| | SVMAHQK, |
| (SEQ ID NO: 41) | |
| | TVMATQK, |
| (SEQ ID NO: 42) | |
| | NVMAQPK, |
| (SEQ ID NO: 43) | |
| | NVMVSCQK, |
| (SEQ ID NO: 44) | |
| | NMMVSCQK, |
| (SEQ ID NO: 45) | |
| | NVMGSCQK, |
| (SEQ ID NO: 46) | |
| | NVLVSSQK, |
| (SEQ ID NO: 47) | |
| | NVMGSYQK, |
| (SEQ ID NO: 48) | |
| | NMMVA-QK, |
| (SEQ ID NO: 49) | |
| | NNIQSKK, |
| (SEQ ID NO: 50) | |
| | NNIQTKK, |
| (SEQ ID NO: 51) | |
| | NNVQTKK, |
| (SEQ ID NO: 52) | |
| | NNIQIKK, |
| (SEQ ID NO: 53) | |
| | NNIHTKK, |
| (SEQ ID NO: 54) | |
| | NTMASSTSK, |
| (SEQ ID NO: 55) | |
| | NTMASS-SK, |
| (SEQ ID NO: 56) | |
| | NTMASNPSK, |
| (SEQ ID NO: 57) | |
| | NTMATNPSK, |
| (SEQ ID NO: 58) | |
| | NTMAANPSK, |
| (SEQ ID NO: 59) | |
| | NTMSSQSSK, |
| (SEQ ID NO: 60) | |
| | NTMASNPPK, |
| (SEQ ID NO: 61) | |
| | NTMAPNPSK, |
| (SEQ ID NO: 62) | |
| | NTMASNSAK, |
| (SEQ ID NO: 63) | |
| | NTMANNSSK, |
| (SEQ ID NO: 64) | |
| | KNSLERTK, |
| (SEQ ID NO: 65) | |
| | KNSLEQTK, |
| (SEQ ID NO: 66) | |
| | KNSFERTK, |

In the above, “−” refers to an amino acid that is absent, i.e. not present.

In an embodiment, the degradation signal peptide may comprise or consist of a (sub)sequence represented by formula I according to one or more embodiments described in this specification, followed by a (sub)sequence represented by formula II according to one or more embodiments described in this specification.

The degradation signal peptide may comprise or consist of a sequence represented by formula III

X₁X₂VGWPPX₃X₄X₅X₆X₇X₈X₉X₁₀X₁₁X₁₂ Formula III

wherein X₁ is Q or absent;

X₂ is absent, V, I, A, or L;

X₃ is V, I, L, G, or A;

X₄ is R, C, or K;

X₅ is N or S;

X₆ is Y, F or W;

X₇ is R or K;

X₈ is K or R;

X₉ is N, S, T, K, or R;

X₁₀ is M, I, V, N, S, T, or L;

X₁₁ is M, I, L, V, S, or T; and

X₁₂ is T, A, V, G, Q, H, S, L, F, or I.

The degradation signal peptide may comprise or consist of a sequence represented by formula IV

X₁X₂VGWPPX₃X₄X₅X₆X₇X₈X₉X₁₀X₁₁X₁₂X₁₃X₁₄X₁₅X₁₆X₁₇X₁₈ Formula IV

wherein

X₁ is Q or absent;

X₂ is absent, V, I, A, or L;

X₃ is V, I, L, G, or A;

X₄ is R, C, or K;

X₅ is N or S;

X₆ is Y, F, or W;

X₇ is R or K; and

X₈ is K or R;

X₉ is N, S, T, K, or R;

X₁₀ is M, I, V, N, S, T, or L;

X₁₁ is M, I, L, V, S, or T;

X₁₂ is T, A, V, G, Q, H, S, L, F, or I;

X₁₃ is absent, Q, N, H, T, S, A, E, P, I, or L;

X₁₄ is absent, Q, P, C, S, Y, K, N, R, or T;

X₁₅ is absent, K, Q, T, P, S, N, or R;

X₁₆ is absent, S, N, T, K, P, or A; and

X₁₇ is absent, S, G, A, P, E, T, N, K, or R;

X₁₈ is absent, S, E, T, G, or N;

or it may be a degradation signal peptide functionally and/or structurally equivalent thereto.

The sequence represented by formula III may be selected from the following (i.e. the degradation signal peptide may comprise or consist of an amino acid sequence selected from the following), or the degradation signal peptide may be a degradation signal peptide functionally and/or structurally equivalent thereto:

| (SEQ ID NO: 67) | |
|---|---|
| | QVVGWPPVRNYRKNMMT, |
| (SEQ ID NO: 68) | |
| | QVVGWPPVRNYRKNIMT, |
| (SEQ ID NO: 69) | |
| | QVVGWPPVRNYRKNIIT, |
| (SEQ ID NO: 70) | |
| | QVVGWPPVRNYRKNVMA, |
| (SEQ ID NO: 71) | |
| | QVVGWPPVRNYRKNIMA, |
| (SEQ ID NO: 72) | |
| | QVVGWPPVRNYRKSVMA, |
| (SEQ ID NO: 73) | |
| | QVVGWPPVRNYRKTVMA, |
| (SEQ ID NO: 74) | |
| | QVVGWPPVRSYRKNIMA, |
| (SEQ ID NO: 75) | |
| | QVVGWPPVRSYRKNVMV, |
| (SEQ ID NO: 76) | |
| | QVVGWPPVRSYRKNMMV, |
| (SEQ ID NO: 77) | |
| | QVVGWPPVRSYRKNVMG, |
| (SEQ ID NO: 78) | |
| | QVVGWPPVRSYRKNVLV, |
| (SEQ ID NO: 79) | |
| | QIVGWPPVRSYRKNNIQ, |
| (SEQ ID NO: 80) | |
| | QIVGWPPIRSYRKNNIQ, |
| (SEQ ID NO: 81) | |
| | QIVGWPPVRSYRKNNVQ, |
| (SEQ ID NO: 82) | |
| | QIVGWPPVRSYRKNNIH, |
| (SEQ ID NO: 83) | |
| | QIVGWPPVRSYRKNSIQ, |
| (SEQ ID NO: 84) | |
| | QVVGWPPIRSYRKNTMA, |
| (SEQ ID NO: 85) | |
| | QVVGWPPIRSYRKNTMS, |
| (SEQ ID NO: 86) | |
| | QVVGWPPIRSFRKNTMA, |
| (SEQ ID NO: 87) | |
| | QVVGWPPVCSYRRKNSL, |
| (SEQ ID NO: 88) | |
| | QAVGWPPVCSYRRKNSL, |
| (SEQ ID NO: 89) | |
| | QVVGWPPVRSYRRKNSF. |

Extending the degradation signal peptide to the PB1 domain of IAAs, which in AtIAA7 starts at AA position 124 of SEQ ID NO: 1, may significantly reduce depletion efficiency. Corresponding positions at which the PB1 domain may be considered to start in other AtIAAs are position 92 in SEQ ID NO: 2 (AtIAA3), 110 of SEQ ID NO: 3 (AtIAA17), 110 of SEQ ID NO: 4 (AtIAA14), 76 of SEQ ID NO: 5 (AtIAA5), and/or 199 of SEQ ID NO: 6 (AtIAA8). Therefore excluding sequences starting from these positions and/or corresponding AAs from the degradation signal peptide may be desirable, as it may achieve improved depletion efficiency. In other words, the polynucleotide and/or the nucleotide sequence encoding the degradation signal peptide may not comprise a sequence encoding the PB1 domain or a portion thereof. Said portion thereof may comprise or consist of a stretch of at least 1, or at least 2, or at least 3, or at least 4 first amino acids of the PB1 domain.

The amino acid sequence of the degradation signal peptide may therefore, in some embodiments, not comprise a (sub)sequence starting at amino acid residues corresponding to positions

124 of SEQ ID NO: 1 (AtIAA7),

92 of SEQ ID NO: 2 (AtIAA3),

110 of SEQ ID NO: 3 (AtIAA17),

110 of SEQ ID NO: 4 (AtIAA14),

76 of SEQ ID NO: 5 (AtIAA5), or

199 of SEQ ID NO: 6 (AtIAA8),

or a sequence at least 80%, or at least 85%, or at least 90%, or at least 95%, or 100% identical to said sequence. In other words, the C-terminal part of the degradation signal peptide may not extend to the amino acid residues corresponding to these positions and optionally to the amino acid residues following them.

In other words, in an embodiment, the amino acid sequence of the degradation signal peptide ends at a residue corresponding to an amino acid residue in the range of amino acid residues

98-123, or 99-123, or 100-123, or 101-122 of SEQ ID NO: 1 (AtIAA7),

80-91, or 81-91, or 82-91, or 83-90 of SEQ ID NO: 2 (AtIAA3),

98-109, or 99-109, or 100-109, or 101-108 of SEQ ID NO: 3 (AtIAA17),

92-109, or 93-109, or 94-109, or 95-108 of SEQ ID NO: 4 (AtIAA14),

69-75, or 70-75, or 71-75, or 72-74 of SEQ ID NO: 5 (AtIAA5), or

181-198, or 182-198, or 183-198, or 184-197 of SEQ ID NO: 6 (AtIAA8).

In this context, the phrase “ends at a residue” may be understood such that said residue (at which the sequence ends) is the last residue of the amino acid sequence of the degradation signal peptide. The amino acid sequence of the degradation signal peptide may thus be understood as comprising at least a partial sequence of the sequence set forth in the corresponding SEQ ID preceding the residue at which the sequence ends. Said residue may be followed by other amino acid residue(s) and/or sequence(s), for example one forming a part of a linker, a tag, a target polypeptide or protein, a moiety capable of associating with a target polypeptide or protein, or any other suitable polypeptide or protein.

In an embodiment, the residue at which the sequence ends does not necessarily have to be the exact amino acid residue of the corresponding SEQ ID NO: 1-6, but it may also be e.g. a conservative amino acid substitution thereof. Various examples of such residues, substitutions and sequences are described in this specification.

In the context of this specification, the phrase of the format “aa:s (or AA:s) (i.e. amino acid residues) 84-98 of SEQ ID NO: 1” may be understood as referring to amino acid residues at positions 84-98 of SEQ ID NO: 1, i.e. amino acid residues corresponding to those at positions 84-98 of SEQ ID NO: 1.

The degradation signal peptide may (but does not necessarily) further comprise an additional preceding subsequence.

The additional preceding subsequence may comprise or consist of a sequence starting at an amino acid residue (position) in the range of amino acid residues corresponding to amino acid residues at positions 1-83, or 1-82, or 1-81, or 35-83, or 35-82, or 35-81, of SEQ ID NO: 1 (AtIAA7), or a sequence at least 80%, or at least 85%, or at least 90%, or at least 95% identical thereto. The additional preceding subsequence may immediately precede the core sequence or be linked thereto via a linker, for example any linker described in this specification. The additional preceding subsequence may thus end at an amino acid residue at position 83, 82 or 81 of SEQ ID NO: 1.

The additional preceding subsequence may comprise or consist of a sequence starting at an amino acid residue (position) in the range of amino acid residues corresponding to amino acid residues at positions 1-65, or 1-64, or 1-63, or 37-65, or 37-64, or 37-63, of SEQ ID NO: 2 (AtIAA3), or a sequence at least 80%, or at least 85%, or at least 90%, or at least 95% identical thereto. The additional preceding subsequence may immediately precede the sequence or be linked thereto via a linker, for example any linker described in this specification. The additional preceding subsequence may thus end at an amino acid residue at position 65, 64 or 63 of SEQ ID NO: 2.

The additional preceding subsequence may comprise or consist of a sequence starting at an amino acid residue (position) in the range of amino acid residues corresponding to amino acid residues at positions 1-83, or 1-82, or 1-81, or 31-83, or 31-82, or 31-81, of SEQ ID NO: 3 (AtIAA17), or a sequence at least 80%, or at least 85%, or at least 90%, or at least 95% identical thereto. The additional preceding subsequence may immediately precede the sequence or be linked thereto via a linker, for example any linker described in this specification. The additional preceding subsequence may thus end at an amino acid residue at position 83, 82 or 81 of SEQ ID NO: 3.

The additional preceding subsequence may comprise or consist of a sequence starting at an amino acid residue (position) in the range of amino acid residues corresponding to amino acid residues at positions 1-77, or 1-76, or 1-75, or 30-77, or 30-76, or 30-75, of SEQ ID NO: 4 (AtIAA14), or a sequence at least 80%, or at least 85%, or at least 90%, or at least 95% identical thereto. The additional preceding subsequence may immediately precede the sequence or be linked thereto via a linker, for example any linker described in this specification. The additional preceding subsequence may thus end at an amino acid residue at position 77, 76 or 75 of SEQ ID NO: 4.

The additional preceding subsequence may comprise or consist of a sequence starting at an amino acid residue (position) in the range of amino acid residues corresponding to amino acid residues at positions 1-54, or 1-53, or 1-52, or 35-54, or 35-53, or 35-52, of SEQ ID NO: 5 (AtIAA5), or a sequence at least 80%, or at least 85%, or at least 90%, or at least 95% identical thereto. The additional preceding subsequence may immediately precede the sequence or be linked thereto via a linker, for example any linker described in this specification. The additional preceding subsequence may thus end at an amino acid residue at position 54, 53 or 52 of SEQ ID NO: 5.

The additional preceding subsequence may comprise or consist of a sequence starting at an amino acid residue (position) in the range of amino acid residues corresponding to amino acid residues at positions 1-166, or 1-165, or 1-164, or 35-166, or 35-165, or 35-164 of SEQ ID NO: 6 (AtIAA8), or a sequence at least 80%, or at least 85%, or at least 90%, or at least 95% identical thereto. The additional preceding subsequence may immediately precede the sequence or be linked thereto via a linker, for example any linker described in this specification. The additional preceding subsequence may thus end at an amino acid residue at position 166, 165 or 164 of SEQ ID NO: 6.

In an embodiment, the degradation signal peptide may comprise or consist of a (sub)sequence represented by formula I according to one or more embodiments described in this specification, optionally followed by a (sub)sequence represented by formula II according to one or more embodiments described in this specification, and an additional preceding subsequence according to one or more embodiments described in this specification.

In an embodiment, the degradation signal peptide may comprise or consist of a (sub)sequence represented by formula I according to one or more embodiments described in this specification, (optionally) followed by a (sub)sequence represented by formula II according to one or more embodiments described in this specification, and preceded by an additional preceding subsequence according to one or more embodiments described in this specification.

The amino acid sequence of the degradation signal peptide may comprise or consist of a sequence
starting at an amino acid residue in the range of amino acid residues corresponding to amino acid residues at positions 1-84, or 1-83, or 1-82, or 1-81, or 35-83, or 35-82, or 35-81, of SEQ ID NO: 1 (AtIAA7), and ending at a residue corresponding to an amino acid residue in the range of amino acid residues at positions 98-123, or 99-123, or 100-123, or 101-122 of SEQ ID NO: 1 (AtIAA7);
starting at an amino acid residue in the range of amino acid residues corresponding to amino acid residues at positions 1-66, or 1-65, or 1-64, or 1-63, or 37-65, or 37-64, or 37-63, of SEQ ID NO: 2 (AtIAA3), and ending at a residue corresponding to an amino acid residue in the range of amino acid residues at positions 80-91, or 81-91, or 82-91, or 83-90 of SEQ ID NO: 2 (AtIAA3);
starting at an amino acid residue in the range of amino acid residues corresponding to amino acid residues at positions 1-84, or 1-83, or 1-82, or 1-81, or 31-83, or 31-82, or 31-81, of SEQ ID NO: 3 (AtIAA17), and ending at a residue corresponding to an amino acid residue in the range of amino acid residues at positions 98-109, or 99-109, or 100-109, or 101-108 of SEQ ID NO: 3 (AtIAA17);
starting at an amino acid residue in the range of amino acid residues corresponding to amino acid residues at positions 1-78, or 1-77, or 1-76, or 1-75, or 30-77, or 30-76, or 30-75, of SEQ ID NO: 4 (AtIAA14), and ending at a residue corresponding to an amino acid residue in the range of amino acid residues at positions 92-109, or 93-109, or 94-109, or 95-108 of SEQ ID NO: 4 (AtIAA14);
starting at an amino acid residue in the range of amino acid residues corresponding to amino acid residues at positions 1-55, or 1-54, or 1-53, or 1-52, or 34-54, or 34-53, or 34-52, of SEQ ID NO: 5 (AtIAA5), and ending at a residue corresponding to an amino acid residue in the range of amino acid residues at positions 69-75, or 70-75, or 71-75, or 72-74 of SEQ ID NO: 5 (AtIAA5);
starting at an amino acid residue in the range of amino acid residues corresponding to amino acid residues at positions 1-167, or 1-166, or 1-165, or 1-164, or 107-166, or 107-165, or 107-164 of SEQ ID NO: 6 (AtIAA8), and ending at a residue corresponding to an amino acid residue in the range of amino acid residues at positions 181-198, or 182-198, or 183-198, or 184-197 of SEQ ID NO: 6 (AtIAA8);

or sequences at least 75%, or at least 80%, or at least 85%, or at least 90%, or at least 95%, or 100% identical thereto;

or it may be a degradation signal peptide functionally and/or structurally equivalent thereto.

In the above embodiments, the amino acid sequence of the degradation signal peptide may be a continuous sequence (a continuous series of amino acid residues) thus forming a part of SEQ ID NO: 1, 2, 3, 4, 5 or 6, or be a sequence at least 80%, or at least 85%, or at least 90%, or at least 95%, or 100% identical to said sequence. Thus a sequence starting at an amino acid residue in the range of amino acid residues corresponding to amino acid residues at specific position(s) and ending at a residue corresponding to an amino acid residue in the range of amino acid residues at specific position(s) may be understood as also comprising the sequence of the respective SEQ ID NO between the starting and ending residues.

For example, the sequence starting at an amino acid residue in the range of amino acid residues corresponding to amino acid residues at positions 1-84 of SEQ ID NO: 1 (AtIAA7), and ending at a residue corresponding to an amino acid residue in the range of amino acid residues at positions 98-123 of SEQ ID NO: (AtIAA7), may be understood as being a continuous sequence extending from an amino acid residue in the range of amino acid residues corresponding to amino acid residues at positions 1-84 of SEQ ID NO: 1 to a residue corresponding to an amino acid residue in the range of amino acid residues at positions 98-123 of SEQ ID NO: 1. Said continuous sequence thus forms a continuous series of at least the amino acid residues 84-98 of SEQ ID NO: 1. It may, at least in some embodiments, extend further along the sequence SEQ ID NO: 1 towards the N-terminus at positions 1-83 and/or towards the C-terminus at positions 99-123.

The length of the additional preceding subsequence and/or the total length of the degradation signal peptide is not particularly limited. For example, degradation signal peptides having a sequence corresponding to amino acid residues 35-104, 37-104, 37-101, 37-98, 52-104, 76-104, 80-104, and 82-104 of SEQ ID NO: 1 may exhibit similar depletion efficiencies. A relatively short degradation signal peptide may be desirable e.g. for simpler constructions and fusions and/or for steric reasons, but a longer degradation signal peptide may also be contemplated.

In an embodiment, the amino acid sequence of the degradation signal peptide comprises a sequence that is at least 75%, or at least 80%, or at least 85%, or at least 90%, or at least 95%, or 100% identical to a sequence corresponding to amino acid residues at positions 84-98 of SEQ ID NO: 1 (AtIAA7). In other embodiments, the sequence may be at least 75%, or at least 80%, or at least 85%, or at least 90%, or at least 95%, or 100% identical to a sequence corresponding to amino acid residues at positions 83-98, or 82-98, or 83-99, or 82-99, or 83-100, or 82-100, or 83-101, or 82-101, or 83-102, or 82-102, or 83-103, or 82-103, or 83-104, or 82-104 of SEQ ID NO: 1 (AtIAA7); or it is a degradation signal peptide functionally and/or structurally equivalent thereto. Such degradation signal peptides exhibit relatively high depletion efficiencies.

In an embodiment, the amino acid sequence of the degradation signal peptide does not comprise a sequence corresponding to amino acid residues at positions 124-132 or 124-167 of SEQ ID NO: 1. In other words, the amino acid sequence of the degradation signal peptide does not comprise a continuous sequence corresponding to amino acid residues at positions 124-132 or 124-167 of the sequence set forth in SEQ ID NO: 1

In an embodiment, the amino acid sequence of the degradation signal peptide comprises a sequence that is at least 75%, or at least 80%, or at least 85%, or at least 90%, or at least 95%, or 100% identical to a sequence corresponding to amino acid residues at positions 66-80 of SEQ ID NO: 2 (AtIAA3); or it is a degradation signal peptide functionally and/or structurally equivalent thereto. In other embodiments, the sequence may be at least 75%, or 80%, or 85%, or 90%, or 95% identical to a sequence corresponding to amino acid residues at positions 65-80, or 64-80, or 65-81, or 64-81, or 65-82, or 64-82, or 65-83, or 64-83, or 65-84, or 64-84, or 65-85, or 64-85, or 65-86, or 64-86 of SEQ ID NO: 2 (AtIAA3); or it is a degradation signal peptide functionally and/or structurally equivalent thereto.

In an embodiment, the amino acid sequence of the degradation signal peptide comprises a sequence that is at least 75%, or at least 80%, or at least 85%, or at least 90%, or at least 95%, or 100% identical to a sequence corresponding to amino acid residues at positions 84-98 of SEQ ID NO: 3 (AtIAA17); or it is a degradation signal peptide functionally and/or structurally equivalent thereto. In other embodiments, the sequence may be at least 75%, or 80%, or 85%, or 90%, or 95% identical to a sequence corresponding to amino acid residues at positions 83-98, or 82-98, or 83-99, or 82-99, or 83-100, or 82-100, or 83-101, or 82-101, or 83-102, or 82-102, or 83-103, or 82-103, or 83-104, or 82-104 of SEQ ID NO: 3 (AtIAA17); or it is a degradation signal peptide functionally and/or structurally equivalent thereto.

In an embodiment, the amino acid sequence of the degradation signal peptide comprises a sequence that is at least 75%, or at least 80%, or at least 85%, or at least 90%, or at least 95%, or 100% identical to a sequence corresponding to amino acid residues at positions 78-92 of SEQ ID NO: 4 (AtIAA14); or it is a degradation signal peptide functionally and/or structurally equivalent thereto. In other embodiments, the sequence may be at least 75%, or at least 80%, or at least 85%, or at least 90%, or at least 95%, or 100% identical to a sequence corresponding to amino acid residues at positions 77-92, or 76-92, or 77-93, or 76-93, or 77-94, or 76-94, or 77-95, or 76-95, or 77-96, or 76-96, or 77-97, or 76-97, or 77-98, or 76-98 of SEQ ID NO: 4 (AtIAA14); or it is a degradation signal peptide functionally and/or structurally equivalent thereto.

In an embodiment, the amino acid sequence of the degradation signal peptide comprises a sequence that is at least 75%, or at least 80%, or at least 85%, or at least 90%, or at least 95%, or 100% identical to a sequence corresponding to amino acid residues at positions 55-69 of SEQ ID NO: 5 (AtIAA5); or it is a degradation signal peptide functionally and/or structurally equivalent thereto. In other embodiments, the sequence may be at least 75%, or at least 80%, or at least 85%, or at least 90%, or at least 95%, or 100% identical to a sequence corresponding to amino acid residues at positions 54-69, or 53-69, or 54-70, or 53-70, or 54-71, or 53-71, or 54-72, or 53-72, or 54-73, or 53-73, or 54-74, or 53-74, or 54-75, or 53-75 of SEQ ID NO: 5 (AtIAA5); or it is a degradation signal peptide functionally and/or structurally equivalent thereto.

In an embodiment, the amino acid sequence of the degradation signal peptide comprises a sequence that is at least 75%, or at least 80%, or at least 85%, or at least 90%, or at least 95%, or 100% identical to a sequence corresponding to amino acid residues at positions 167-181 of SEQ ID NO: 6 (AtIAA8); or it is a degradation signal peptide functionally and/or structurally equivalent thereto. In other embodiments, the sequence may be at least 75%, or at least 80%, or at least 85%, or at least 90%, or at least 95%, or 100% identical to a sequence corresponding to amino acid residues at positions 166-181, or 165-181, or 166-182, or 165-182, or 166-182, or 165-182, or 166-183, or 165-183, or 166-184, or 165-184, or 166-185, or 165-185, or 166-186, or 165-186, or 166-187, or 165-187 of SEQ ID NO: 6 (AtIAA8); or it is a degradation signal peptide functionally and/or structurally equivalent thereto.

The residue of the amino acid sequence corresponding to position 101 of SEQ ID NO: 1, to position 83 of SEQ ID NO: 2, to position 101 of SEQ ID NO: 3, to position 95 of SEQ ID NO: 4, to position 72 of SEQ ID NO: 5, or to position 184 of SEQ ID NO: 6 may be K, R or Q. In an embodiment, said residue may be K or R.

In an embodiment, the amino acid sequence of the degradation signal peptide is other than a sequence corresponding to amino acid residues at positions 63-109 of SEQ ID NO: 3 (AtIAA17), and/or a sequence corresponding to AAs 68-132 of AtIAA17 (SEQ ID NO: 3) (i.e. mAID degron of 65 amino acids, referred herein to as miniAID).

A polypeptide or protein is also disclosed, the polypeptide or protein comprising the degradation signal peptide encoded by the polynucleotide according to one or more embodiments described in this specification.

The polypeptide or protein may be a fusion polypeptide or a fusion protein comprising the degradation signal peptide fused to a target polypeptide or protein, for example directly or via a linker sequence. The degradation signal peptide may be fused to the N-terminal part or to the C-terminal part of the target polypeptide or protein. Various suitable linker sequences, for example flexible linkers, are available to a skilled person. The skilled person may also select, test and optimize the linker sequence and the fusion junction(s) such that it does not interfere with the function of the degradation signal peptide and/or of the target polypeptide or protein.

Other ways of attaching the degradation signal peptide to the target polypeptide or protein may be contemplated.

An expression cassette comprising the polynucleotide according to one or more embodiments described in this specification is also disclosed. The expression cassette may comprise one or more sequences for expression in a host cell.

The polynucleotide may be operatively linked to the one or more sequences for expression in a host cell, and/or wherein the expression cassette comprises one or more sequences for introducing the nucleotide sequence encoding the degradation signal peptide and/or the polynucleotide to a host genome, optionally for fusing the polynucleotide to a target gene. The nucleotide sequence encoding the degradation signal peptide and/or polynucleotide, or parts thereof, and the target gene may thus form a genetic fusion, such that the degradation signal peptide and the target polypeptide or protein are translated into a single fusion polypeptide or protein.

The one or more sequences for expression in a host cell may include one or more sequences that are sufficient to drive the expression of the nucleotide sequence encoding the degradation signal peptide, the polynucleotide and/or of the fusion formed by the nucleotide sequence encoding the degradation signal peptide and/or polynucleotide and the target gene in a suitable host cell or organism, such as a promoter sequence. The term “promoter” may refer to a polynucleotide, for example DNA, which may be recognized and bound (directly or indirectly) by a DNA-dependent RNA-polymerase during initiation of transcription. A promoter may include a transcription initiation site, and binding sites for transcription initiation factors and RNA polymerase, and may comprise various other sites (e.g., enhancers), at which gene expression regulatory proteins may bind. Various promoters, terminator sequences and other regulatory sequences for driving and/or regulating the expression in a host cell are available and may be selected based on e.g. the host cell or transgenic organism, the target polypeptide or protein, the desired specificity of expression and other considerations. In embodiments in which the nucleotide sequence encoding the degradation signal peptide and/or the polynucleotide is fused to a target gene, the (native) promoter and/or other sequences for the expression and/or regulation of the target gene may function as driving the expression of the fusion of the polynucleotide and the target gene.

The polynucleotide or the expression cassette may further comprise e.g. a linker sequence linking the nucleotide sequence encoding the degradation signal peptide and the target gene or the nucleotide sequence encoding the target polypeptide or protein. The skilled person may also select, test and optimize the linker sequence and the fusion junction(s) such that they do not interfere with the function of the degradation signal peptide and/or of the target polypeptide or protein.

The polynucleotide, expression cassette and/or vector may further comprise a sequence encoding a target polypeptide or protein, such that the target polypeptide or protein is fused to the degradation signal peptide. The target polypeptide or protein may be fused to the degradation signal peptide via a linker sequence or directly. The degradation signal peptide may be fused to the N-terminal part or to the C-terminal part of the target polypeptide or protein. The fusion may naturally be optimized e.g. by selecting a terminal part at which the fusion is most effective and/or functional.

The polynucleotide may be operatively linked to one or more sequences for expression in a host cell and/or comprises one or more sequences for introducing the nucleotide sequence encoding the degradation signal peptide and/or polynucleotide to a gene of a host genome, thereby fusing the polynucleotide to a target gene. The host cell and host genome may be any host cell or host genome described in this specification. For example, the polynucleotide may comprise homology arms for CRISPR/Cas9-mediated homology-directed repair (HDR). The homology arms may flank the part of the polynucleotide which is intended for integrating into the host genome.

Thus the entire polynucleotide or a part thereof may be integrated into the host genome. For example, at least the nucleotide sequence encoding the the degradation signal peptide may be introduced or integrated into the host genome. However, other parts may be introduced or integrated into the host genome as well, for example a nucleotide sequence encoding the target polypeptide or protein or a moiety capable of associating with the target polypeptide or protein, the one or more sequences for expression in the host cell, a nucleotide sequence encoding a label or a tag, and/or a nucleotide sequence encoding a linker.

The nucleotide sequence encoding the degradation signal peptide, and/or the polynucleotide, or one or more parts of the polynucleotide may codon optimized for expression in a host cell, for example in a mammalian cell. Examples of codon optimized sequences are shown in Table 1 below. In an embodiment, the nucleotide sequence encoding the degradation signal peptide is selected from the following: SEQ ID NOs: 90, 91, 92, 93, or is at least 75%, or at least 80%, or at least 85%, or at least 90%, or at least 95%, or at least 98%, or at least 99%, identical thereto. The polynucleotide, the expression cassette and/or the vector may further comprises a sequence encoding a moiety capable of associating with a target polypeptide or protein, such that the moiety is fused to the degradation signal peptide, optionally via a linker sequence. The moiety may be a polypeptide, protein, domain, tag or other fusion partner. The moiety may be capable of binding the target polypeptide or protein directly or indirectly, or it may be otherwise capable of physically associating with the target polypeptide or protein, such that when auxin or auxin analogue binds to a functional auxin perceptive protein and induces at least a partial depletion of the target polypeptide or protein by causing the auxin perceptive protein to bind to the degradation signal peptide. For example, it may be a binding agent, binding moiety or binding domain capable of binding the target polypeptide or another moiety fused to the target polypeptide. As an example, the moiety may be a nanobody or other antibody or antibody fragment. The moiety may be capable of binding e.g. a GFP (green fluorescent protein), other fluorescent protein, or other tag fused to the target polypeptide or protein. An exemplary embodiment is described in Daniel et al., Nature Communications 2018, 9, 3297 (DOI: 10.1038/s41467-018-05855-5), which describes an AID-nanobody capable of binding GFP-tagged proteins.

The moiety fused to the degradation signal peptide, optionally via a linker sequence, may then associate with, for example by binding directly or indirectly, the target polypeptide or protein, or otherwise bring the degradation signal peptide in physical proximity or contact of the target polypeptide or protein. In the presence of a functional auxin perceptive protein and auxin or an auxin analogue, the functional auxin perceptive protein may then bind to the degradation signal peptide, recruit an E2 ubiquitin conjugating enzyme and polyubiquitylate the fusion protein comprising the moiety as well as the target polypeptide or protein. The moiety associating with the target polypeptide or protein may thereby result in their rapid degradation by the proteasome.

The polynucleotide and/or expression cassette may further comprise e.g. a sequence encoding a label or a tag, such as a label or a tag for detecting the fusion polypeptide or fusion protein comprising the degradation signal peptide fused to a target polypeptide or protein and to the label or tag. For example, a sequence encoding a fluorescent polypeptide or protein may be suitable for detecting and possibly e.g. quantifying the fusion polypeptide or protein and/or its depletion.

Well suited fluorescent proteins include e.g. mEGFP and mCherry, but various other fluorescent proteins, labels and tags may be available, for example SNAP-Tag® and CLIP-Tag®, HaloTag® tags and various others. The skilled person may select, test and optimize the label or tag and the sequence encoding it, and in particular the fusion junction(s) and/or linker(s), such that they do not interfere with the function of the degradation signal peptide and/or of the target polypeptide or protein.

They may also be selected and/or optimized such that they are optimal for the functionality of the degradation signal peptide and/or of the target polypeptide or protein. For example, mEGFP has been found to work well, in particular when the C-terminus of the degradation signal peptide is fused to the N-terminus of mEGFP, optionally via a linker. An exemplary embodiment is shown in SEQ ID NO: 94, in which a degradation signal peptide of aa:s 37-104 of AtIAA7 (SEQ ID NO: 1) is linked via a two-AA linker (SG) to mEGFP. In other words, in an embodiment, the sequence encoding a label, such as a fluorescent polypeptide or protein, is a sequence encoding mEGFP (for example, mEGFP as set forth in SEQ ID NO: 95). The sequence encoding mEGFP may be fused to the C-terminus of the degradation signal peptide, optionally via a linker, but it may alternatively be fused to the N-terminus of the degradation signal peptide, again optionally via a linker.

The sequence encoding the label or tag may be fused to the target polypeptide or protein and/or to the degradation signal peptide in various different orientations, for example so that the degradation signal peptide and the label or tag are fused to the N-terminal end of the target polypeptide or protein, or so that the degradation signal peptide and the label or tag are fused to the C-terminal end of the target protein or polypeptide. The order may depend e.g. on the specific target protein or polypeptide, on the label or tag, on the host cell and/or other considerations.

The polynucleotide and/or the expression cassette may comprise one or more sequences for targeting and optionally integrating the nucleotide sequence encoding the degradation signal peptide and/or the polynucleotide to a desired site in the host genome, for example to a safe harbor site. An example would be e.g. the AAVS1/Safe harbor locus, to which it may be targeted using e.g. the CRISPR/Cas9 technology, other knock-in technology or other targeting/genomic integration technology. Other safe harbour sites and integration technologies may also be contemplated, depending e.g. on the host cell and genome, for example the CCR5 site, the murine Rosa26 locus and/or an ortholog thereof. The one or more sequences for targeting may include e.g. HR targeting sequences or homology arms for tagging an endogenous locus. For example, the polynucleotide may be a synthetic DNA polynucleotide or a PCR fragment.

However, it is not always necessary to generate a knock-in, but simply insert one or more copies of the expression cassette and/or the polynucleotide, for example for overexpression of the fusion polypeptide or protein.

In this context, the host cell and/or the host genome may again be any host cell or host genome described in this specification.

A vector is further disclosed, the vector comprising the polynucleotide according to one or more embodiments described in this specification and/or the expression cassette according to one or more embodiments described in this specification.

In the context of this specification, the term “vector” may be understood as referring to a polynucleotide produced by recombinant DNA techniques for delivering genetic material into a cell and optionally integrating at least a portion thereof in the genome of the cell. As is well known in the art, it may refer to a plasmid, a cosmid, an artificial chromosome, a cloning vector, an expression vector or any other suitable vector. The vector may be a DNA vector, but RNA vectors may also be contemplated.

It may, alternatively or additionally, be possible to introduce a polypeptide or protein comprising the degradation signal peptide encoded by the polynucleotide according to one or more embodiments described in this specification into a host cell or a host organism.

In the vector, the polynucleotide may be operatively linked to one or more sequences for expression in a host cell, and/or wherein the vector comprises one or more sequences for introducing the polynucleotide to a host genome, thereby fusing the polynucleotide to a target gene.

The vector may, as a skilled person knows, further comprise other parts or sequences, for example a backbone, sequences required for replication of the vector or for selection, etc.

The vector may comprise one or more sequences for targeting the polynucleotide sequence encoding the degradation signal peptide to a desired site in the host genome, for example to a safe harbor site. An example would be e.g. the AAVS1/Safe harbor locus, to which it may be targeted using e.g. the CRISPR/Cas9 technology. The one or more sequences for targeting may include e.g. HR targeting sequences. The vector may thus be e.g. a HR targeting (donor) vector.

The vector may, additionally or alternatively, suitable for transient overexpression.

A system for at least partially depleting a target polypeptide or protein in a host cell is disclosed, the system comprising the polynucleotide according to one or more embodiments described in this specification, the expression cassette according to one or more embodiments described in this specification, and/or the vector according to one or more embodiments described in this specification, and

a second polynucleotide, a second expression cassette and/or a second vector comprising the second polynucleotide, wherein the second polynucleotide encodes a functional auxin perceptive protein capable of binding the degradation signal peptide in the presence of auxin and/or an auxin analogue.

The second polynucleotide and/or the second expression cassette may, in some embodiments, be included in the same polynucleotide or vector as the polynucleotide or expression cassette according to one or more embodiments described in this specification. However, such a polynucleotide or vector may be quite large. Therefore, in other embodiments, the second polynucleotide and/or the second expression cassette may be included in a separate polynucleotide (molecule), expression cassette, or vector.

In the context of this specification, the term “functional auxin perceptive protein” may refer to a polypeptide or protein, or a fragment thereof, which is capable of binding an auxin and/or an auxin analogue. Upon binding the auxin and/or auxin analogue, the functional auxin perceptive protein is capable of binding the degradation signal peptide, thereby targeting the degradation signal peptide and any target polypeptide or protein (and any optional further parts fused thereto) to proteasomal degradation. Examples of such functional auxin perceptive proteins may include e.g. auxin perceptive F-box proteins such as TIR and AFB2 proteins, for example AtAFB2 (accession number NP_566800.1, SEQ ID NO: 96), OsTIR1, MnTIR1 (accession number XP_010112739.2, SEQ ID NO: 97), GhAFB2 (accession number XP_016709605.1, SEQ ID NO: 98), NcAFB2 (accession number A0A1J3CY17, SEQ ID NO: 99), and/or MnAFB2 (accession number XP_010096050.1, SEQ ID NO: 100). Also other proteins that are e.g. at least 80%, or at least 85%, or at least 90%, or at least 95%, or 100% identical to AtAFB2 (SEQ ID NO: 96), OsTIR1, MnTIR1 (SEQ ID NO: 97), GhAFB2 (SEQ ID NO: 98), NcAFB2 (SEQ ID NO: 99), MnAFB2 (SEQ ID NO: 100), AtTIR1, a derivative thereof, such as AtTIR1 or any other TIR1 F79G mutant, or (functional) fragments thereof, may be contemplated.

The functional auxin perceptive protein may be AtAFB2 (SEQ ID NO: 96), a functional auxin perceptive protein having a sequence that is at least 80%, or at least 85%, or at least 90%, or at least 95%, or 100% identical to AtAFB2 (SEQ ID NO: 96), or a polypeptide or a protein comprising at least one stretch that is at least 80%, or at least 85%, or at least 90%, or at least 95%, or 100% identical to a continuous stretch of at least 60 amino acids of AtAFB2 (SEQ ID NO: 96), or a fragment thereof. Such functional auxin perceptive proteins, such as AtAFB2, have been found to provide good depletion efficiencies and relatively low constitutive depletion together with one or more embodiments of the degradation signal peptide described in this specification.

The second polynucleotide encoding the functional auxin perceptive protein may be codon optimized, for example for expression in a mammalian host cell or other host cell as described in this specification. Examples of the second polynucleotide encoding the functional auxin perceptive protein may include the following, or a sequence at least 80%, or at least 85%, or at least 90%, or at least 95%, or at least 99% identical, or 100% identical thereto:

AtAFB2 (SEQ ID NO: 96, aa residues 1-575) (an exemplary codon optimized cDNA sequence set forth in SEQ ID NO: 101);

MnTIR1 (SEQ ID NO: 97) (an exemplary codon optimized cDNA sequence set forth in SEQ ID NO: 102);

GhAFB2 (SEQ ID NO: 98) (an exemplary codon optimized cDNA sequence set forth in SEQ ID NO: 103);

NcAFB2 (SEQ ID NO: 99) (an exemplary codon optimized cDNA sequence set forth in SEQ ID NO: 104);

MnAFB2 (SEQ ID NO: 100) (an exemplary codon optimized cDNA sequence set forth in SEQ ID NO: 105).

The functional auxin perceptive protein may exhibit minimal basal degradation. Such functional auxin perceptive proteins may include, for example,
AtAFB2 (SEQ ID NO: 96), a functional auxin perceptive protein having a sequence that is at least 80%, or at least 85%, or at least 90%, or at least 95%, or 100% identical to AtAFB2 (SEQ ID NO: 96), or a polypeptide or a protein comprising at least one stretch that is at least 80%, or at least 85%, or at least 90%, or at least 95%, or 100% identical to a continuous stretch of at least 60 amino acids of AtAFB2 (SEQ ID NO: 96); and/or

a derivative of a TIR1 protein, such as AtTIR1, containing the F79G mutation. The F79G mutation in AtTIR1 has been described e.g. in Uchida et al., Nature Chemical Biology 2018, 14, 299-305.

A functional auxin perceptive protein may be considered to exhibit minimal basal degradation, when at most 50%, or at most 40%, or at most 30%, or at most 20%, or at most 15%, of the target polypeptide or protein is constitutively depleted in the absence of an inducer. The extent of the constitutive depletion may be determined e.g. as mentioned above or as shown in the Examples of the present specification.

The second polynucleotide, the second expression cassette and/or the second vector may further comprise a nucleotide sequence encoding a localization sequence for directing the localization of the functional auxin perceptive protein. The localization sequence may be a subcellular localization sequence. The exact localization sequence may be selected e.g. depending on the host cell or genome and/or the localization of the target polypeptide or protein. The localization sequence may thus be fused to the functional auxin perceptive protein. For example, the localization sequence may be a nuclear localization sequence.

The term “nuclear localization sequence”, “NLS” or “nuclear localization signal” may be understood as an amino acid sequence that directs the protein to which it is fused, in this case the functional auxin perceptive protein, to the nucleus of the host cell. Examples of NLSs may include weak NLS (MycA1, AAAKRVKLD, SEQ ID NO: 106), strong NLS (Myc, PAAKRVKLD, SEQ ID NO: 107), and/or the SV40 large T antigen NLS (PKKKRKV, SEQ ID NO: 108), although other NLSs may also be contemplated, e.g. depending on the host cell, the functional auxin perceptive protein and other considerations.

A kit is disclosed, comprising the polynucleotide according to one or more embodiments described in this specification, the expression cassette according to one or more embodiments described in this specification, the vector according to one or more embodiments described in this specification, and/or the system according to one or more embodiments described in this specification. The kit may further comprise instructions for use. The kit may be suitable for performing the method(s) according to one or more embodiments described in this specification. The kit may further comprise other components and/or reagents, for example a solvent, a buffer, an enzyme (for example, an enzyme for cloning purposes and/or for polymerase chain reaction), transfection reagent(s), one or more primers (e.g. for polymerase chain reaction for cloning purposes), etc.

A host cell comprising the nucleotide sequence encoding the degradation signal peptide and/or the polynucleotide according to one or more embodiments described in this specification, the expression cassette according to one or more embodiments described in this specification, the vector according to one or more embodiments described in this specification, and/or the system according to one or more embodiments described in this specification is also disclosed. The host cell may be any host cell described in this specification, e.g. an animal cell or a fungal cell. For example, the host cell may be a mammalian cell, for example a human, murine, bovine, ovine, porcine, feline, canine, equine, or primate cell; a nematode cell; or an insect cell. In an embodiment, the host cell is other than a plant cell.

A transgenic organism stably transformed or transfected with the polynucleotide according to one or more embodiments described in this specification is also disclosed. The transgenic organism may therefore contain the nucleotide sequence encoding the degradation signal peptide and/or the polynucleotide stably integrated into its genome. The transgenic organism may, alternatively or additionally, be stably transformed or transfected with the expression cassette according to one or more embodiments described in this specification, the vector according to one or more embodiments described in this specification, and/or the system according to one or more embodiments described in this specification. The transgenic organism may be an animal or a fungus, a mammal, e.g. a rodent such as a mouse or a rat, a fish, an insect, or a nematode, such as C. elegans, or any other host described in this specification. The term “transgenic organism” may be understood as referring to an organism in which nucleotide sequence encoding the degradation signal peptide and/or the polynucleotide or expression cassette according to one or more embodiments described in this specification is stably integrated into the genome. The term may also encompass the progeny of the transgenic organism which is stably transformed or transfected.

A method for at least partially depleting a target polypeptide or protein in a host cell is also disclosed. The method may comprise

introducing the polynucleotide according to one or more embodiments described in this specification, the expression cassette according to one or more embodiments described in this specification, the vector according to one or more embodiments described in this specification and/or the system according to one or more embodiments described in this specification to the host cell, such that the nucleotide sequence encoding the degradation signal peptide and/or the polynucleotide forms a fusion with a target gene encoding the target polypeptide or protein or a moiety capable of associating with a target polypeptide or protein, the fusion encoding a fusion protein comprising the degradation signal peptide and the target polypeptide or protein or the moiety capable of associating with the target polypeptide or protein; or providing the host cell, wherein the nucleotide sequence encoding the degradation signal peptide and/or the polynucleotide forms a fusion with a target gene encoding the target polypeptide or protein or a moiety capable of associating with the target polypeptide or protein, the fusion encoding a fusion protein comprising the degradation signal peptide and the target polypeptide or protein or the moiety capable of associating with the target polypeptide or protein;

expressing the fusion protein in the host cell;

expressing a functional auxin perceptive protein in the host cell; and

introducing an auxin or an auxin analogue to the host cell, such that the auxin or the auxin analogue binds to the functional auxin perceptive protein and induces at least a partial depletion of the fusion protein or of the target polypeptide or protein by causing the auxin perceptive protein to bind to the degradation signal peptide. The host cell may be any host cell described in this specification, for example an animal cell or a fungal cell.

The method may be performed at a temperature suitable for the growth and/or maintenance of the host cell. For example, it may be performed at a temperature of about 37° C., or of 36-38° C.

A method for producing the host cell according to one or more embodiments described in this specification is also disclosed, comprising introducing the polynucleotide according to one or more embodiments described in this specification, the expression cassette according to one or more embodiments described in this specification, the vector according to according to one or more embodiments described in this specification, the polypeptide according to one or more embodiments described in this specification and/or the system according to one or more embodiments described in this specification into the host cell.

In the context of any method described in this specification, the functional auxin perceptive protein may be any functional auxin perceptive protein described in this specification.

In an embodiment, the functional auxin perceptive protein is AtAFB2 (SEQ ID NO: 96), a functional auxin perceptive protein having a sequence that is at least 80%, or at least 85%, or at least 90%, or at least 95%, or 100% identical to AtAFB2 (SEQ ID NO: 96), or a polypeptide or a protein comprising at least one stretch that is at least 80%, or at least 85%, or at least 90%, or at least 95%, or 100% identical to a continuous stretch of at least 60 amino acids of AtAFB2 (SEQ ID NO: 96).

The method(s) may further comprise introducing a second polynucleotide, a second expression cassette and/or a second vector comprising the second polynucleotide, wherein the second polynucleotide encodes a functional auxin perceptive protein capable of binding the degradation signal peptide in the presence of auxin and/or an auxin analogue into the host cell. The second polynucleotide, the second expression cassette and/or second vector may be any second polynucleotide, any second expression cassette and/or any second vector described in this specification.

The use of the polynucleotide according to one or more embodiments described in this specification, the expression cassette according to one or more embodiments described in this specification, the vector according to one or more embodiments described in this specification, the system according to one or more embodiments described in this specification, or the kit according to one or more embodiments described in this specification for at least partially depleting a target polypeptide or protein in a host cell is also disclosed.

In an embodiment, basal degradation of the target polypeptide or protein is at most 50%, or at most 40%, or at most 30%, or at most 20%, or at most 15%, in the absence of an inducer but optionally in the presence of a functional auxin perceptive protein, the polynucleotide, the polypeptide, the expression cassette, the vector, and/or of the system according to one or more embodiments described in this specification.

Again, the host cell may be any host cell described in this specification, for example an animal cell or a fungal cell.

### EXAMPLES

Reference will now be made in detail to various embodiments, an example of which is illustrated in the accompanying drawings.

The description below discloses some embodiments in such a detail that a person skilled in the art is able to utilize the embodiments based on the disclosure. Not all steps or features of the embodiments are discussed in detail, as many of the steps or features will be obvious for the person skilled in the art based on this specification.

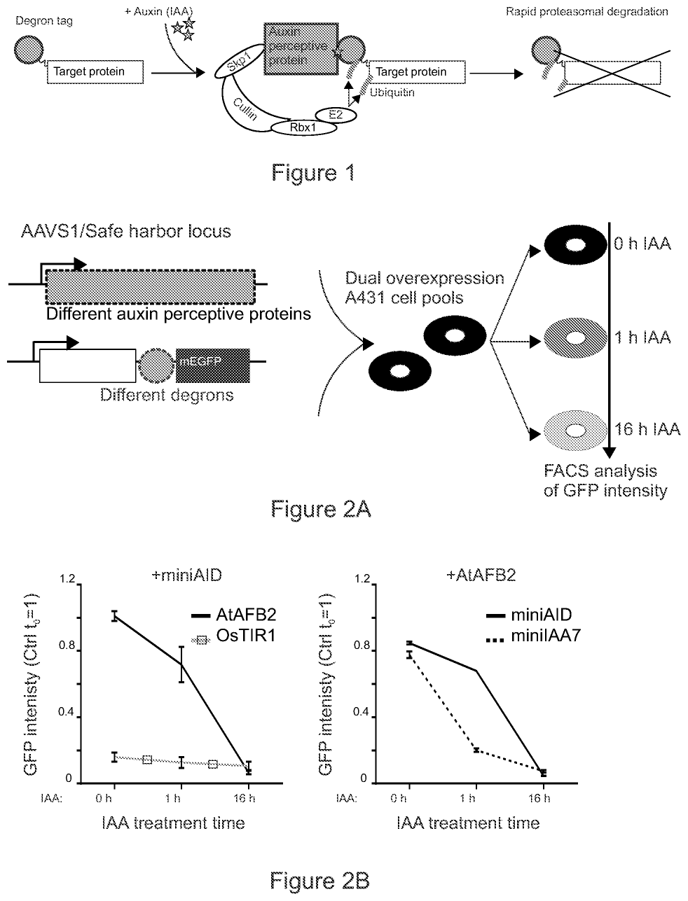

FIG. 1 illustrates schematically the function of the degradation signal peptide, i.e. degron tag, and at least partially depleting a target polypeptide or protein in a host cell. The auxin-inducible degron (AID) technique controls targeted protein degradation with the small molecule auxin or an auxin analogue. In AID, a degron sequence, i.e. a sequence encoding a degradation signal peptide, is attached to a target polypeptide or protein by genetic fusion.

Alternatively or additionally, the sequence encoding a degradation signal peptide may be attached to a moiety capable of associating with the target polypeptide or protein. For example, the degradation signal peptide may be fused to an anti-GFP nanobody capable of binding to a target polypeptide or protein fused with a GFP moiety. An example of such a system is described in Daniel et al., Nature Communications 2018, 9, 3297 (DOI: 10.1038/s41467-018-05855-5).

Addition of a plant hormone of the auxin class, i.e. an auxin such as 3-acetic acid (IAA) or an analogue thereof, may promote the binding of the degron tag by an auxin perceptive F-box protein TIR1/AFB. An exogenously overexpressed TIR1/AFB forms a functional Skp1-Cullin-F box type E3 ubiquitin ligase (SCF^{TIR1/AFB}) with endogenous subunits conserved in all eukaryotic cells. The auxin-induced binding thus recruits an E2 ubiquitin conjugating enzyme and polyubiquitylates the degron fusion protein, resulting in its rapid degradation by the proteasome.

### Example 1—Construction of a New AID System

Initially seipin, a conserved transmembrane protein in the endoplasmic reticulum (ER) involved in lipid droplet (LD) biogenesis, was targeted for degradation. To rapidly deplete seipin from human A431 cells, an AID system, composed of TIR1 derived from Oryza sativa (OsTIR1) and mAID degron of 65 amino acids corresponding to AAs 68-132 of AtIAA17 (SEQ ID NO: 3), referred herein to as miniAID, was first employed. To this end, the endogenous seipin was homozygously tagged with mAID-mEGFP. However, seipin tagged with a degron termed miniAID (composed of AtIAA17 amino acid residues 68-132) was severely degraded in cells expressing OsTIR1 without IAA addition. Consequently, cells exhibited defective LD biogenesis already before IAA addition, resembling a seipin knockout phenotype (data not shown). The results indicated that AID can deplete seipin efficiently, but that the AID system used suffered from severe constitutive depletion.

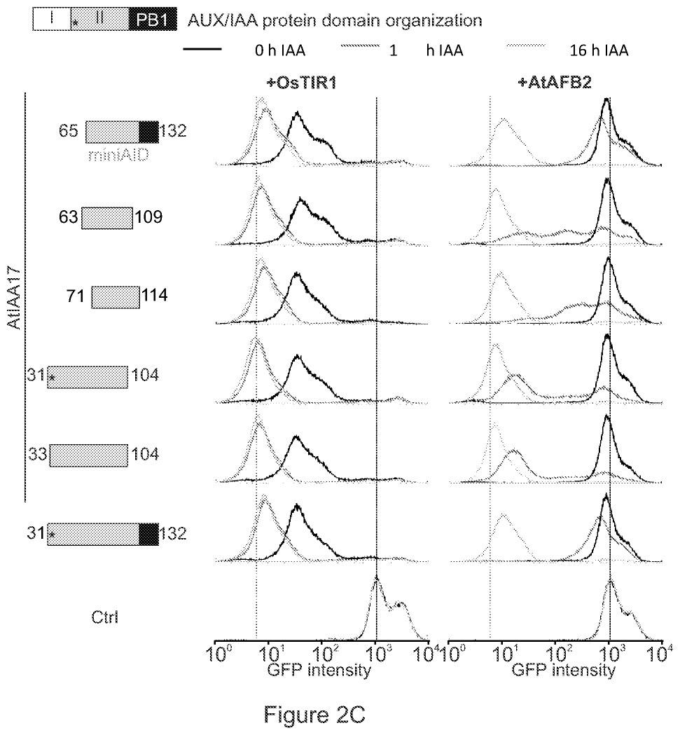

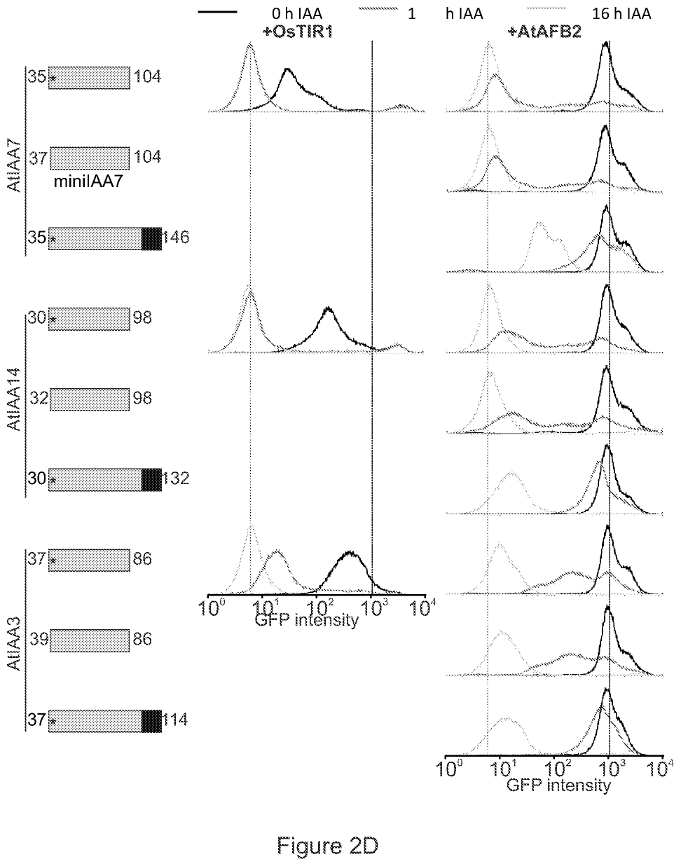

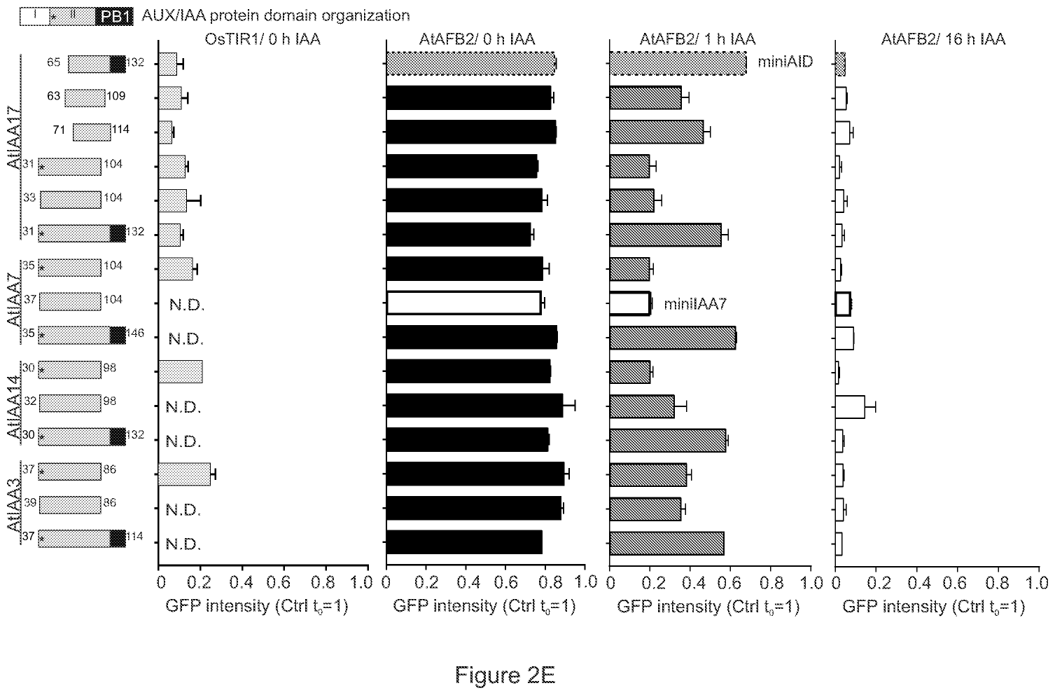

To search for an improved AID system to solve the issue, a pipeline was first established in human A431 cells to screen AID components (FIG. 2A). Various auxin perceptive proteins and degrons were selected for screening. Several TIR1 and AFB2 proteins from different plant species were tested, as well as degrons derived from AtIAA17 (SEQ ID NO: 3), these including amino acid (aa.) 65-132 (miniAID), aa. 62-109, aa. 71-114. All these degrons were included in the screen. AtIAA17 aa.31-104, as well as homolog fragments derived from other AUX/IAA proteins (AtIAA3 (SEQ ID NO: 2), 7 (SEQ ID NO: 1) and 14 (SEQ ID NO: 4)), has been characterized in vitro binding assay and showed the highest IAA binding affinity. These high affinity fragments were tested, assuming higher affinity might translate into more efficient inducible degradation. Degrons with KR dipeptide deletions were tested to see if they would have an effect on enhancing IAA binding affinity, IAA-inducible depletion or as part of a nuclear localization signal. PB1 domain of the AUX/IAA proteins has not been implicated in auxin-inducible degradation, so several other degrons with PB1 domain sequences homologous to miniAID were also tested.

OsTIR1 was first compared to other auxin perceptive proteins, using miniAID as the degron. Arabidopsis thaliana AFB2 (AtAFB2) was identified as the best hit: compared to OsTIR1, it displayed minimal basal depletion with over 5-fold higher target protein level before IAA addition (0 h IAA), and similar auxin-inducible depletion at 16 h IAA treatment (FIG. 2B). However, auxin-inducible depletion with AtAFB2 at 1 h IAA was inefficient. Next, miniAID was compared to other degrons, using either OsTIR1 or AtAFB2. All degrons showed severe basal degradation with OsTIR1 at 0 h IAA but minimal basal degradation with AtAFB2 (FIGS. 2C-E). A degron composed of AtIAA7 (SEQ ID NO: 1) amino acids (aa.) 37-104 (hereafter denoted as ‘miniIAA7’) was identified as an optimal degron in combination with AtAFB2. It dramatically improved auxin-inducible depletion with over 3-fold more efficient protein reduction compared to miniAID at 1 h IAA (FIG. 2B and FIGS. 2D-E). Thus, the improved AID system composed of AtAFB2 and miniIAA7 showed both minimal basal degradation and rapid auxin-inducible depletion.

In both Example 1 and in Example 2 below, A431 cells (ATCC, Cat #CRL-1555) were cultured in DMEM (Lonza), and A549 cells (ATCC, Cat #CCL-185) in F-12 Nutrient Mixture (Gibco), both supplemented with 10% FBS, penicillin/streptomycin (100 U/ml each), L-glutamine (2 mM) at 37° C. in 5% CO₂. Mycoplasma testing was performed regularly using PCR detection. Cells were transfected at 80-95% confluence using Lipofectamine LTX with PLUS Reagent (Life Technologies), typically with 1.0 μg plasmid(s) per 1.0 μl of PLUS reagent, 2.0 μl of Lipofectamine LTX and 4.0×10⁵ (A431) or 3.0×10⁵ (A549) cells in a 12-well. Indole-3-acetic acid sodium (IAA, Santa Cruz, sc-215171) was prepared at 100× in H₂O (10 mg/ml), aliquoted, stored at −20° C. and used within 2 days after thawing.

Construction of AAVS1 Site Specific Integration Vectors

AAVS1 safe harbour locus site-specific integration was conducted with CRISPR/Cas9-mediated homology-directed repair (HDR). A donor vector was generated by assembling PCR amplified fragments by restriction digestion and ligation. The resulting vector contained two homology arms (from A431 genomic DNA) flanking an overexpression cassette with puromycin selection marker (from pEFIRES-P) on a plasmid backbone (from pGL3-basic). This donor vector was designated as pSH-EFIRES-P and used to express different auxin perceptive proteins. A second donor vector was generated by changing the puromycin selection marker on the first vector with blasticidin selection marker. This donor vector was designated as pSH-EFIRES-B and used to express seipin-mEGFP with different degrons. A third vector co-expressing Cas9 and a sgRNA (both derived from PX458, Addgene #48138) was designated as pCas9-sgRNA. The vector was inserted with two sgRNAs targeting AAVS1 safe harbour locus (sgAAVS1-1 target sequence: ACCCCACAGTGGGGCCACTA GGG (SEQ ID NO: 109); sgAAVS1-2 target sequence: GTCACCAATCCTGTCCCTAG TGG (SEQ ID NO: 110)). Auxin perceptive proteins, except OsTIR1 (Addgene #72835), and degron tags were codon-optimized and synthesized by Genscript (sequences in Table 1). Auxin perceptive proteins were tagged with mCherry through overlap PCR using a 5 aa. linker GGSGG (SEQ ID NO: 111). AtAFB2-mCherry with different NLSs were weak NLS (MycA1, AAAKRVKLD, SEQ ID NO: 106) and strong NLS (Myc, PAAKRVKLD, SEQ ID NO: 107). The three vectors with insertions will be deposited in Addgene.

Screening of Different Auxin Perceptive Proteins and Degrons

OsTIR1 (Addgene #72835) and miniAID for tagging endogenous seipin (Addgene #72825) were gifts from Masato Kanemaki. OsTIR1 was also used as a template for constructing NES-OsTIR1 (OsTIR1 with N-terminus FAK NES2: M-LDLASLIL-SG-OsTIR1 aa. 2-575; the NES peptide sequence LDLASLIL is shown as SEQ ID NO: 112) and OsTIR1-NES (OsTIR1 with C-terminus NES21: OsTIR1 aa. 1-575-IDELLKELADLNLD; the NES21 peptide sequence IDELLKELADLNLD is shown as SEQ ID NO: 113). Other auxin perceptive proteins and degron sequences were codon-optimized and synthesized by Genscript (see Table 1 for synthesized sequences of the degron tags and SEQ ID NO:s 101-105 for codon optimized cDNA sequences for AtAFB2, MnTIR1, GhAFB2, NcAFB2, and MnAFB2, respectively).

Generation of A431 Cell Pools for Screening

A431 cell pools were generated to stably express different combinations of auxin perceptive protein and degron-fused seipin. Cells were cotransfected with a mixture of three vectors composed of pSH-EFIRES-P expressing an auxin perceptive protein, pSH-EFIRES-B expressing a degron-fused seipin, and pCas9-sgAAVS1 at ratio 3:3:4. Transfected cells were passaged 4-6 h after transfection at 1:5 into 6-well plates. On the next day, cells were selected with 1 μg/ml puromycin (Sigma, P8833) for 2 days, and with 5 μg/ml blasticidin (Gibco, A1113904) for 2 days, then with both antibiotics for at least 6 days before using for FACS analysis.

TABLE 1
Degron tags and their codon optimized cDNA sequences.

| | | Amino | | |
|---|---|---|---|---|
| | | acid | | |
| Origin | Identifier | range | Amino acid sequence | cDNA sequence (codon optimized) |
| AtIAA17 | NP_171921.1 | 31-132 | KRGF-SETVDLKLNLNNEPA | AAGCGGGGCTTCAGCGAGACCGTGGACCTGA |
| | | | NKEGSTTHDVVTFDSKEKSA | CAGCTGAAC-TGAACAATGAGCCCGCCAATA |
| | | | CPKD-PAKPPAKA-QVVGWP | AGGAGGGCTCCACCACACACGAC-GTGGTGA |
| | | | PVRSYRKNVMVSCQKSSGGP | CATTTGATTCTAAGGAGAA-GAGCGCCTGCC |
| | | | E-AAAFVKVSMDGAPYLRKI | CTAAGGACCCCGCAAAGCCAC-CTGCCAAGG |
| | | | D-LRMYK (SEQ ID NO: | CACAGGTGGTGGGATGGCCACCCGTGCGGTC |
| | | | 114) | C-TACAGAAAGAACGTGATGGTGTCTTGTCA |
| | | | | GAA-GAGCTCCGGCGGCCCCGAGGCAGCAGC |
| | | | | CTTCGTGAAGGTGTC-TATGGACGGCGCCCC |
| | | | | TTACCTGAGGAAGATCGATCTGCG-CATGTA |
| | | | | TAAG (SEQ ID NO: 90) |
| AtIAA7 | NP_001326465.1 | 35-146 | KRGFSETVDLM-LNLQSNKE | AAGAGGGGCTTCTCTGAGACCGTGGACCTGA |
| | | | GSVDLKNVSAV-PKEKTTLK | TGCTGAACCTGCAG-TCCAATAAGGAGGGCT |
| | | | DPSKPPAKA-QVVGWPPVRN | CTGTGGATCTGAAGAAC-GTGAGCGCCGTGC |
| | | | YRKNMMTQQKTSSGAEEASS | CTAAGGAGAAGACCACAC-TGAAGGACCCAT |
| | | | EKAGNFGG-GAAGAGLVKVS | CCAAGCCCCCTGCCAAGGCACAGGTGGTGG- |
| | | | MDGAPYL-RKVDLKMYK | GATGGCCACCCGTGCGGAACTACAGAAAGAA |
| | | | (SEQ ID NO: 115) | TATGATGACCCAG-CAGAAGACAAGCTCCGG |
| | | | | CGCAGAGGAGGCATCTAGCGA-GAAGGCCGG |
| | | | | CAATTTTGGAGGAGGAGCAGCAGGAGCAG-G |
| | | | | ACTGGTGAAGGTGTCCATGGACGGAGCACCA |
| | | | | TACCTGCG-GAAGGTGGATCTGAAGATGTAT |
| | | | | AAG (SEQ ID NO: 91) |
| AtIAA14 | ADL70642.1 | 30-132 | KRGF-SETVDLKLNLQSNKQ | AAGAGGGGCTTCTCTGAGACCGTGGACCTGA |
| | | | GHVDLNTNGAPKEKTFLKDP | AGCTGAACCTG-CAGAGCAATAAGCAGGGCC |
| | | | SKP-PAKA-QVVGWPPVRNY | ACGTGGATCTGAACAC-CAATGGCGCCCCTA |
| | | | RKNVMAN-QKSGEAEEAMSS | AGGAGAAGACATTTCTGAAGGACCCAAGCA |
| | | | GGGT-VAFVKVSMDGAPYL- | A-GCCCCCTGCCAAGGCACAGGTGGTGGGAT |
| | | | RKVDLKMYT (SEQ ID | GGCCACCCGTGCG-GAACTACAGAAAGAATG |
| | | | NO: 116) | TGATGGCCAAC-CAGAAGTCCGGCGAGGCAG |
| | | | | AGGAGGCAATGAGCTCCGGCG-GAGGCACCG |
| | | | | TGGCCTTCGTGAAGGTGTCTATGGACGGAGC |
| | | | | AC-CATACCTGCGGAAGGTGGATCTGAAGAT |
| | | | | GTATACA (SEQ ID NO: 92) |
| AtIAA3 | NP_171920.1 | 37-114 | KRVLSTDTEKEIES-SSRKT | AAGCGGGTGCTGTCCACCGACACAGAGAAGG |
| | | | ETSP-PRKAQIVGWPPVRSY | AGATCGA-GAGCTCCTCTAGGAAGACCGAGA |
| | | | RKNNIQSKKNESEHEGQGIY | CATCCCCACCTAG-GAAGGCACAGATCGTGG |
| | | | VKVSMDGAPYLRKIDLSCYK | GATGGCCACCCGTGCGGTCTTACAGAAA-GA |
| | | | (SEQ ID NO: 117) | ACAATATCCAGAGCAAGAAGAACGAGTCCGA |
| | | | | GCAC-GAGGGCCAGGGCATCTATGTGAAGGT |
| | | | | GTCTATGGAC-GGCGCCCCCTACCTGAGGAA |
| | | | | GATCGATCTGAGCTGCTATAAG (SEQ ID |
| | | | | NO: 93) |

FACS Analysis

For FACS analysis, cells were seeded at 1:5 (for A431) or 1:3 (for A549) into 6-well plate in medium without selection on day 0. On day 1, medium was changed to 2 ml fresh medium without (for 0 h and 1 h IAA samples) or with (for 16 h IAA samples) 0.5 mM IAA. On day 2, the 1 h samples were supplemented with 0.5 ml medium containing 2.5 mM IAA (final 0.5 mM) and incubated for 1 h at 37° C. After treatment, cells were detached with 0.5 ml trypsin at 37° C. for 5-8 min (A549) or 8-12 min (A431), put on ice, and transferred to 1.5 ml Eppendorf tubes containing 0.5 ml serum-free CO₂ independent medium (Gibco). The cell suspensions were centrifuged at 4° C., resuspended in 0.3 ml ice-cold serum-free FluoroBrite DMEM (Gibco) and stored on ice prior to FACS analysis. FACS analysis was performed on a BD Influx cell sorter (BD Biosciences-US) with 100 μm nozzle at 4-8° C. using BD FACS Sortware. Cells were gated with SSC, FSC and trigger pulse width for singlets and 100 000 cells were analyzed for each sample. GFP was excited with 488 nm laser and detected with 530/40 detector; mCherry was excited with 561 nm lasers and detected with 615/20 detector. Data was analyzed with BD FACS Sortware. Background subtracted mean fluorescence intensity was used for analysis.

### Example 2—Testing of AtAFB2-miniIAA7 System for Rapidly Depleting Endogenous Proteins and Revealing Acute Phenotypes

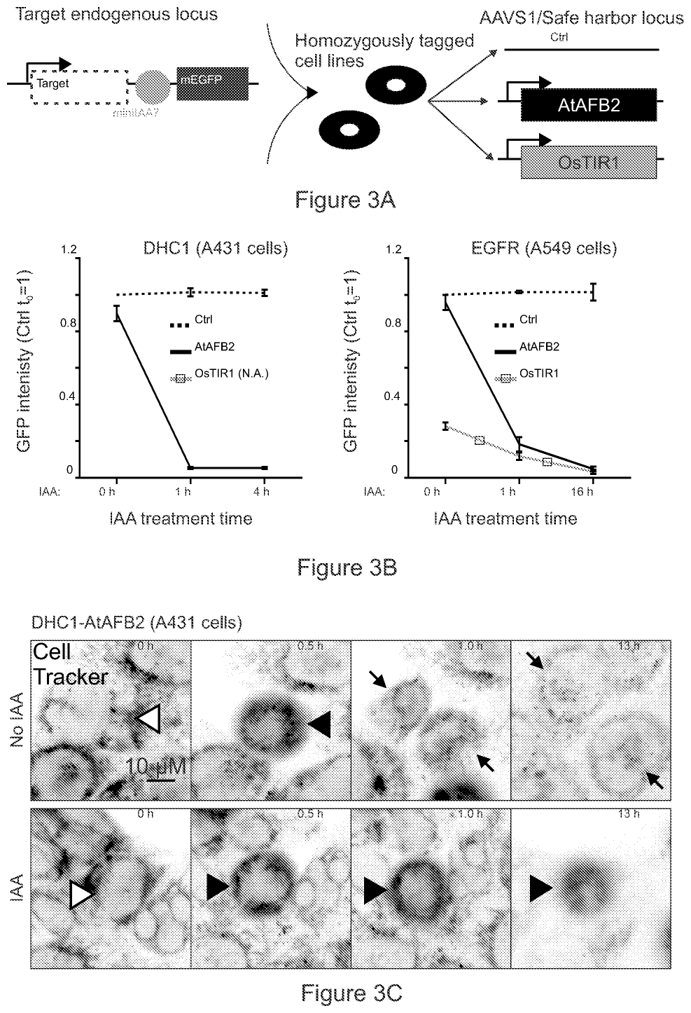

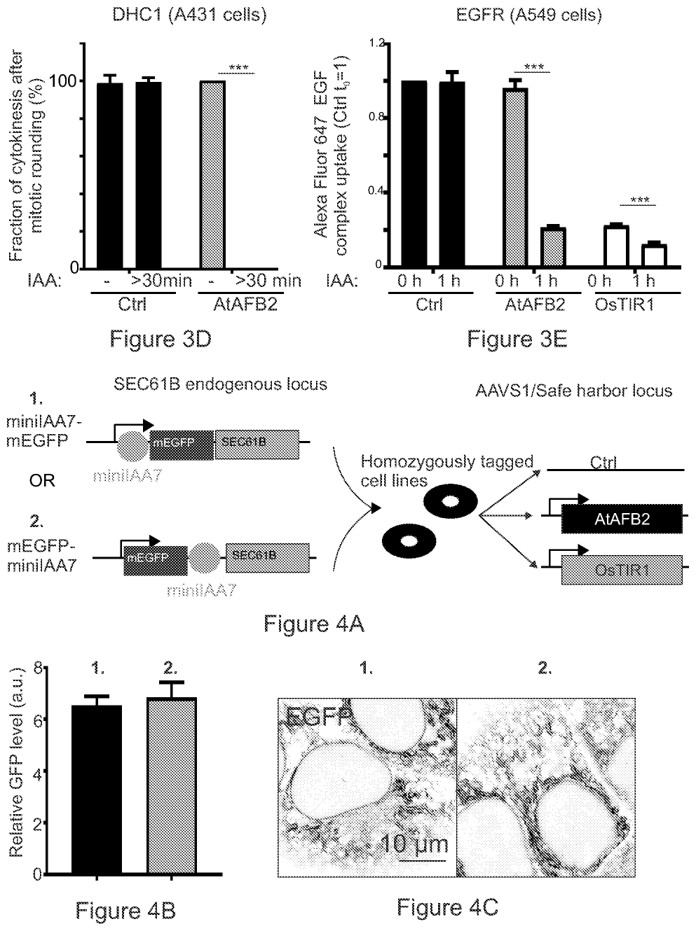

The AtAFB2-miniIAA7 system (FIG. 3A) was tested for rapidly depleting endogenous proteins and revealing acute phenotypes. Dynein heavy chain (DHC1) and epidermal growth factor receptor (EGFR) were chosen as the first targets. DHC1 is an essential protein that could not be rapidly depleted by using the OsTIR1-miniAID system in a previous study (Natsume et al., 2016, Cell Rep. 15, 210-218). EGFR is a transmembrane receptor with a canonical function in EGF uptake that can be acutely assessed after protein depletion. Endogenous target loci were tagged homozygously in human cells with miniIAA7-mEGFP through Cas9-mediated homology-directed repair (FIG. 3A). DHC1 was tagged homozygously but it was only possible to tag EGFR heterozygously in A431 cells, likely due to its high copy numbers in this cell type (data not shown). However, homozygous tagging of EGFR was achieved in human A549 cells. AtAFB2, or OsTIR1 for comparison, was then expressed by introducing it into the AAVS1 loci of the homozygous knock-in clones. The parental cell lines not expressing an auxin perceptive protein were used as controls (FIG. 3A). It was found that both DHC-AtAFB2 (DHC1 homozygously tagged with miniIAA7-mEGFP and AtAFB2 expressed) and EGFR-AtAFB2 cells showed minimal basal degradation at 0 h IAA, and efficient auxin-inducible depletion at 1 h IAA and at longer times (FIG. 3B). In comparison, DHC1-OsTIR1 cells died out during selection, and EGFR-OsTIR1 cells showed severe basal depletion at 0 h IAA (FIG. 3B).

Next it was assessed whether rapid auxin-inducible depletion revealed acute phenotypes. FIG. 3C shows time-lapse images of A431-DHC1 cells with or without cell division after mitotic cell rounding. Open arrowhead: cells before mitosis; filled arrowhead: cells undergoing mitotic rounding; arrow: cells after cell division. In DHC1-AtAFB2 cells, the fraction of mitotic rounding cells that completed cell division was 0% after IAA addition for 30 min, compared to 100% without IAA addition (FIG. 3D). In EGFR-AtAFB2 cells, EGF uptake was reduced by 75% pending on 1 h IAA treatment, while severe IAA-independent reduction of EGF uptake happened in EGFR-OsTIR1 cells (FIG. 3E). The inducer IAA per se did not affect cell division or EGF uptake as shown in the controls (FIGS. 3D and 3E). Overall, these results demonstrate the improved performance of the AtAFB2-miniIAA7 system in rapidly depleting endogenous proteins and revealing acute phenotypes.

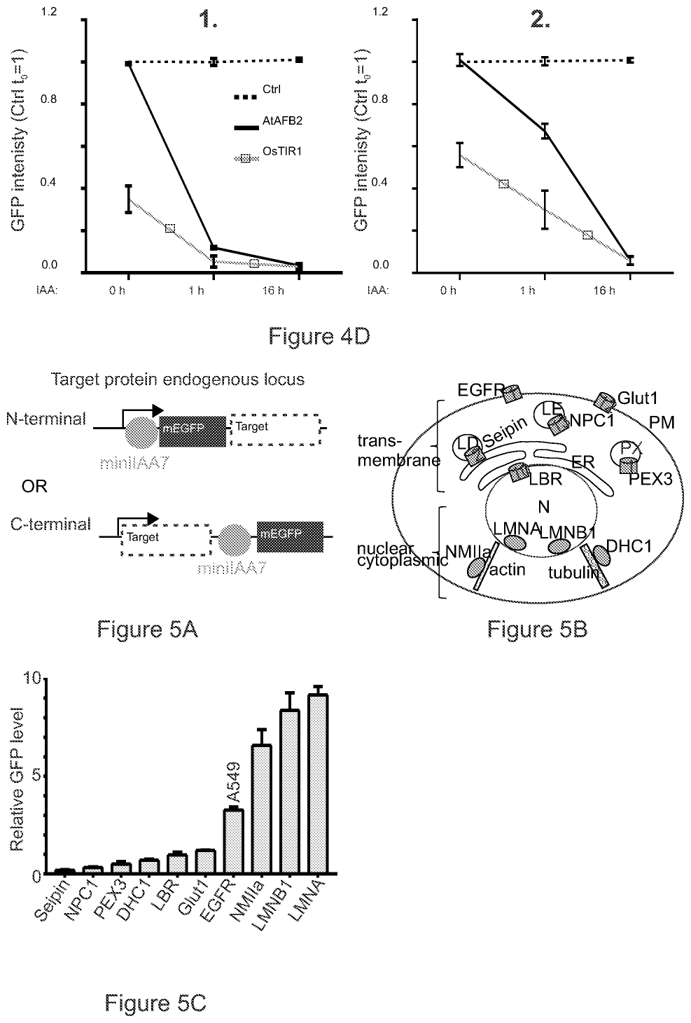

In previous experiments, miniIAA7 was used as a C-terminal tag. Next, miniIAA7 was tagged N-terminally to an endogenous protein. SEC61B was chosen as it is a common target tagged N-terminally through homology-directed repair. It was found that N-terminally tagged SEC61B can be depleted efficiently in 1 h with the AtAFB2-miniIAA7 system (FIGS. 4A-D). Interestingly, the orientation miniIAA7-mEGFP instead of mEGFP-miniIAA7 in the tag provided for optimal depletion kinetics (FIGS. 4A-D). Thus, miniIAA7 works for both N- and C-terminal tagging when using miniIAA7-mEGFP as a fixed unit.

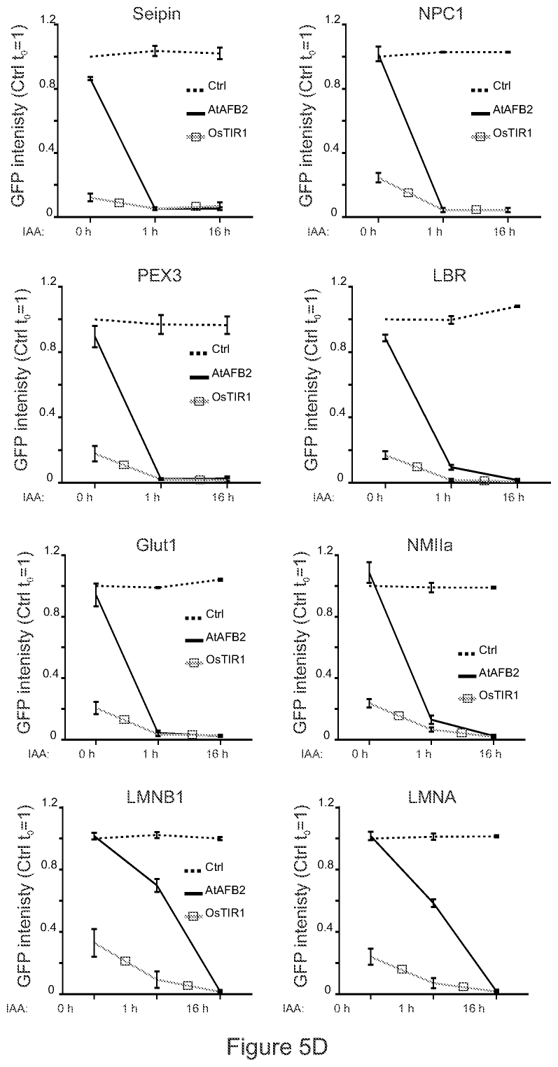

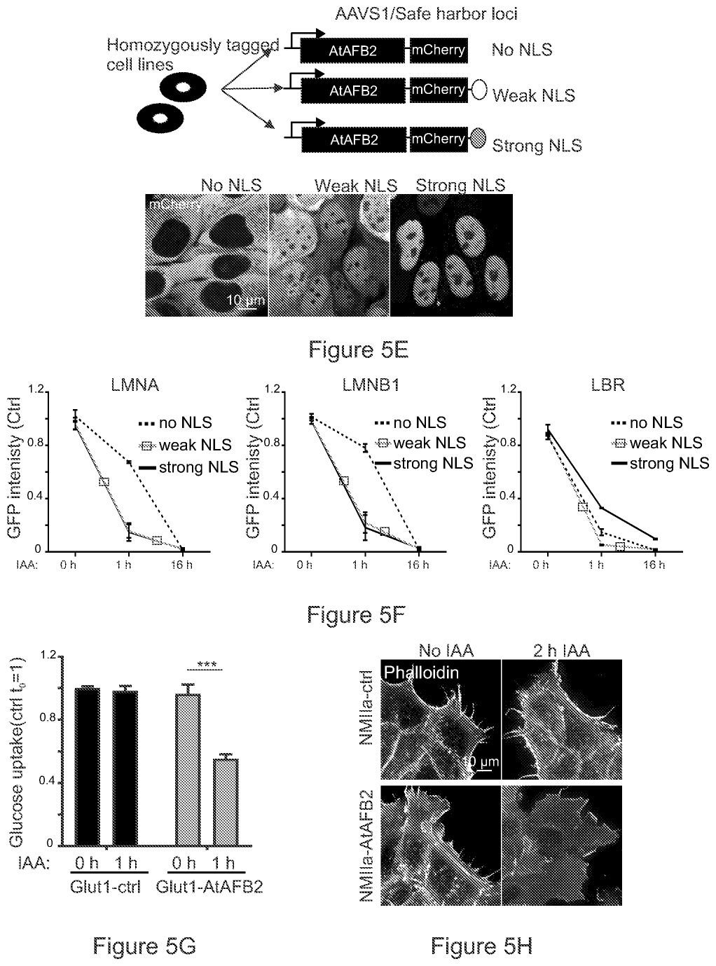

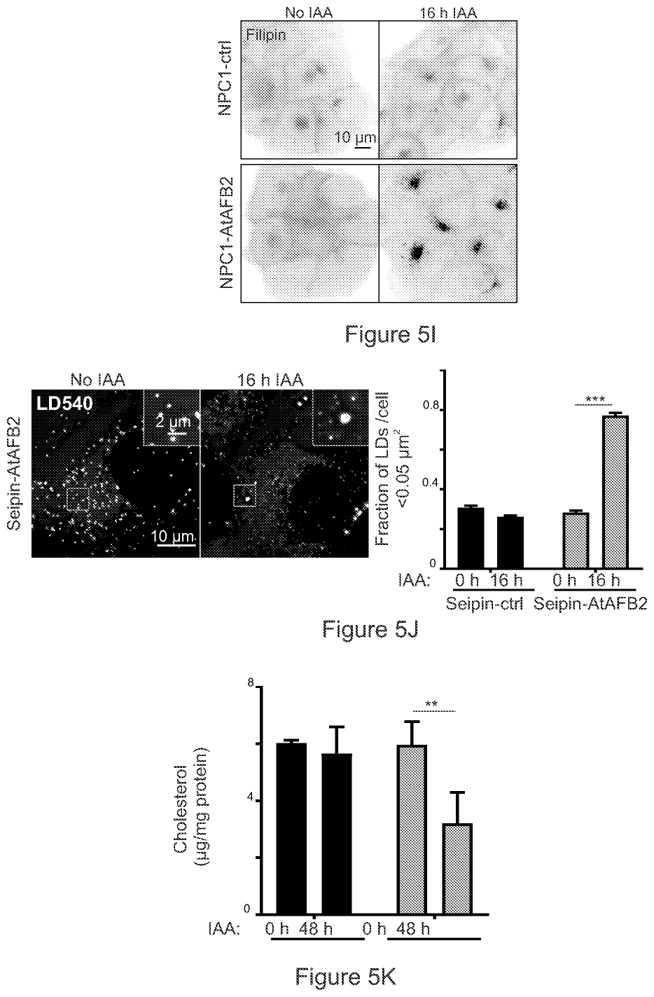

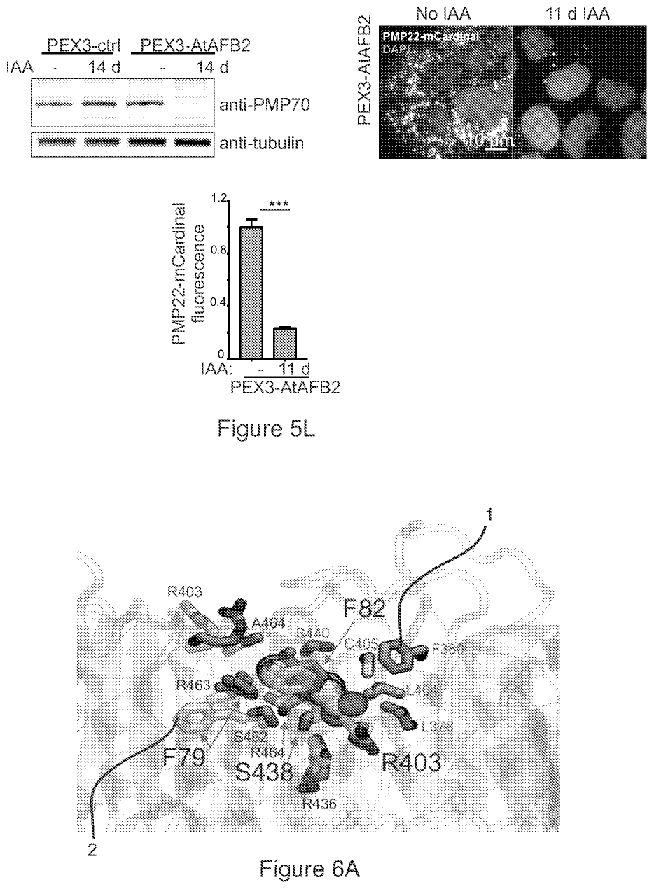

Then the overall performance of the AtAFB2-miniIAA7 system in depleting different endogenous proteins was evaluated. A diverse set of endogenous loci was tagged homozygously with miniIAA7-mEGFP N- or C-terminally (FIG. 5A) and AtAFB2 or OsTIR1 were introduced into the AAVS1 locus (as in FIG. 3A). The target proteins represented different subcellular localizations and a variable number of transmembrane segments, including the original target seipin and a long-lived protein LMNB1 (FIG. 5B). The expression levels of the target proteins in the established cell lines varied by ˜50-fold (ranging from 0.19 for seipin to 9.21 for LMNA; FIG. 5C). When examining the performance of AtAFB2-miniIAA7 system with these targets, it was found that all targets had minimal basal degradation (86-109% levels of control) at 0 h IAA, and were depleted to 2-5% levels at 16 h IAA (FIG. 5D). Notably, the targets showed variable depletion efficiency at 1 h IAA. Non-nuclear targets expressed at low levels (seipin, NPC1, PEX3 and Glut1) were depleted to 2-5%. A non-nuclear target expressed at high level (NMIIa) and a nuclear target expressed at low level (LBR) were also depleted to less than 12%. However, highly expressed nuclear proteins (LMNA and LMNB1) were not as efficiently depleted (FIG. 5D). The OsTIR1-miniIAA7 combination exhibited some basal degradation at 0 h IAA (12-37%) and a depletion efficiency of 2-7% at 16 h IAA with all targets (FIG. 5D).

The depletion of LMNA and LMNB1 was further improved using AtAFB2-miniIAA7 system. Because AtAFB2-mCherry localized predominantly to cytosol (FIG. 5E), the nuclear localization of AtAFB2-mCherry was increased by fusing nuclear localization signals (NLSs) to it (FIG. 5E). Both weak and strong NLSs increased the nuclear localization of AtAFB2-mCherry and substantially improved auxin-inducible depletion of LMNA and LMNB1 at 1 h IAA (FIG. 5E, F). Of note, LBR that is not restricted to the nucleus showed efficient auxin-inducible depletion with the weak but not with the strong NLS construct (FIG. 5F). In summary, these results demonstrate the AtAFB2-miniIAA7 system rapidly depleted all selected endogenous transmembrane, cytoplasmic and nuclear proteins at 1 h with minimal basal degradation. These data also underscore that for rapid protein depletion, the depletion may be improved if AtAFB2 is present at sufficient levels in the compartment where the target protein resides.

It was found that depletion of the endogenous targets with the AtAFB2-miniIAA7 system revealed robust and expected phenotypes as early as 1 h after IAA addition, depending on the functional readouts. These included reduced glucose uptake in Glut1 degron cells (cells with AtAFB2-miniIAA7 system targeting Glut1), massive changes of F-actin structures in NMIIa degron cells, accumulation of cholesterol in late endosomal compartments in NPC1 degron cells, lipid droplet biogenesis defects in seipin degron cells, reduction of cellular cholesterol levels in LBR degron cells, and extensive degradation of peroxisomal membrane proteins in PEX3 degron cells (FIGS. 5G-L).

Construction of Vectors for Endogenous Tagging

Degron tagging of endogenous loci was conducted with CRISPR/Cas9-mediated HDR. Donor vectors with 2 homology arms flanking the degron tag, and Cas9 vectors with specific sgRNAs were constructed for each target. For constructing donor vectors, homology arms were amplified from A431 genomic DNA. MiniIAA7-mEGFP tags were amplified from established templates in the screens above. Overlap PCR was then performed to assemble PCR fragments. Nested PCR primers were used to improve the PCR efficiency and specificity. All PCR amplification steps were performed with Q5 Hot Start High-Fidelity DNA Polymerase (NEB). The PCR fragments were cloned into plasmid backbones using HiFi DNA assembly kit (NEB) or through restriction ligation. Some of the donor vectors were generated by changing the inserts on the established donor vectors through restriction ligation. For constructing Cas9 vectors, target sites were searched manually for -NGG PAM sequence within 18 bp after insertion sites or CCN-PAM within 18 bp before insertion sites. These position restraints were set for both high-efficient integration and for avoiding further mutations after successful HDR. The DHC1 target sites were selected at the 3′-UTR as no target site was available in the searching range. A pCas9/QRVR-sgRNA vector was later constructed through PCR mutagenesis of pCas9-sgRNA to enable use of target sites with -NGA (or TCN-) PAM in the searching range. SgRNAs were synthesized as two unphosphorylated primers, annealed and inserted into BbsI-cut pCas9-sgRNA or pCas9/VRQR-sgRNA vector. Information about endogenous targets and HDR templates is provided in Table 2.

TABLE 2
HDR templates and sgRNAs used

| | | | | HDR | | |
|---|---|---|---|---|---|---|
| | | | | efficiency | | Final |
| | | N or C | Used sgRNA target sequence | (homozy-gous | Length | template/ |
| | Gene | terminal | (Selected for the highest | plus | of HA | plasmid |
| Target | ID | tagging | HDR efficiency) | heterozygous) | (L/R) | assembly |
| MYH9 | 4627 | N | AAGTCACC [symbol] TGGCACAGCAAGCTGCCGAT | 32.4% | 0.92 K/0.67 K | Overlap PCR |
| | | | AAG (SEQ ID NO: 118) | | | (Nested)/Hifi |
| | | | | | | DNA assembly |
| | | | AAGTCACCATGGCACAGCAAGCTGCCGAT | 27.6% | | |
| | | | AAG (SEQ ID NO: 119) | | | |
| LMNA | 4000 | N | CCAACCTGCCGGCCATGGAGACCCCGTCC | 65.8% | 0.94 K/0.88 K | 3 fragments/ |
| | | | C (SEQ ID NO: 120) | | | Hifi DNA |
| | | | | | | assembly |
| LMNB1 | 4001 | N | CCGCC [symbol] TGGCGACTGCGACCCCCGTGCCG | 42.0% | 1.14 K/1.15 K | Overlap PCR |
| | | | (SEQ ID NO: 121) | | | (Nested)/Hifi |
| | | | | | | DNA assembly |
| LBR | 393 | C | CCATACATCTACTAATGCTCTTCTGGCTT | 33.2% and | 1.07 K/0.54 K | Overlap PCR |
| | | | (SEQ ID NO: 122) | 34.1% | | (Nested)/Hifi |
| | | | | | | DNA assembly |
| NPC1 | 4864 | C | TTCTAAATTTCTAGCCCTCTCGCAGGGCA | 41.6% | 1.28 K/1.29 K | Overlap PCR |
| | | | (SEQ ID NO: 123) | | | (Nested)/Hifi |
| | | | | | | DNA assembly |
| EGFR | 1956 | c | GTGAATTTATTGGAGCATGACCACGGAGG | 50.8% and | 0.54 K/1.23 K | Overlap PCR |
| | | | (SEQ ID NO: 124) | 47.3% | | (Nested)/Hifi |
| | | | | (17.9% in | | DNA assembly |
| | | | | A549) | | |
| Glut1 | 6513 | c | TGATTCCCAAGTGTGAGTCGCCCCAGATC | 51.9% | 1.09 K/1.37 K | Overlap PCR |
| | | | ACC (SEQ ID NO: 125) | | | (Nested)/Hifi |
| | | | | | | DNA assembly |
| DHC1 | 1778 | C | GAGTAAACTTTTCTAGCTGCCCCTTTCTG | 20.8% | 1.47 K/1.13 K | Overlap PCR/ |
| | | | TAA-TAGTGAAAGTTGGTAT (SEQ ID | | | Hifi DNA |
| | | | NO: 126) | | | assembly |
| Sec61B | 10952 | N | CATCTCCAATATGGTATGGCGGCCCTTC | 42.0% | 1.02 K/1.52 K | Overlap PCR/ |
| | | | (SEQ ID NO: 127) | | | Restriction |
| | | | | | | digestion |
| | | | | | | ligation |
| Seipin | 26580 | C | ACCTGCTCTAGTTCCTGAAGAAAAGGGGC | 36.7% and | 0.74 K/1.03 K | Overlap PCR/ |
| | | | (SEQ ID NO: 128) | 38.4% | | Restriction |
| | | | | | | digestion |
| | | | | | | ligation |
| PEX3 | 8504 | C | CCCTCAGCAACTGGAGAAATGATTTTTCC | 32.2% | 0.55 K/0.28 K | 3 fragments/ |
| | | | (SEQ ID NO: 129) | | | Hifi DNA |
| | | | | | | assembly |

Generation of Homozygously Tagged Cell Lines

For generation of homozygously tagged cell lines, HDR pools were first generated, followed by FACS enrichment of high GFP expressing cells and limiting dilution in 96-well plates to obtain single clones. Single clones were screened first by fluorescence microscopy for proper GFP expression and subcellular localization, then by genomic PCR to check for homozygous tagging. A detailed protocol is described below.

For generation of HDR pools, A431 or A549 cells in 12-well plates were first transfected with a donor vector (0.6 μg) plus a Cas9/sgRNA vector encoding puromycin resistance gene (0.4 μg). After 4-6 h, cells were passaged into 10 cm dishes. The next day, medium was changed to 1 μg/ml puromycin for 2 days, then to normal medium without puromycin. This procedure eliminated efficiently untransfected cells without selecting for stable puromycin resistant cells. After culturing in normal medium for 4 days, the cells were passaged to fresh medium for 2 days and the resulting cells were considered as the HDR pools. For each target, typically 2-3 sgRNAs were tried in duplicate, and HDR efficiency in the pools was assessed roughly by fluorescence microscopy. HDR pools with the highest efficiency were used for FACS analysis as above, and cells with the highest 1-5% GFP intensity were gated for sorting. The sorted cells were used for single clone isolation with limiting dilution in 96-well plates. For each pool, 10-20 clones were isolated, from which 2-3 clones were picked with fluorescent microscopy for high GFP signal and proper subcellular localization. These clones were further tested for homozygous tagging using genomic PCR. The best sgRNAs and their efficiency in HDR pools analysed by FACS are listed in Table 2.

Generation of Degron Cell Lines

Homozygously tagged single clones were used to generate degron cells overexpressing an auxin perceptive protein. Auxin perceptive proteins were introduced into the AAVS1 safe harbour loci of single clones through Cas9 mediated HDR. Cells were transfected with 0.4 μg pCas9-sgAAVS1 and 0.6 μg pSH-EFIRES-P plasmid encoding an auxin perceptive protein. Transfected cells were passaged at 1:5 after 4-6 h. The next day, cells were selected with 1 μg/ml puromycin for 6 days before passaging for experiments or further culturing. The resulting cell pools were used for FACS analysis and loss-of-function studies without single cloning. 5 μg/ml of puromycin was used occasionally to improve the expression level of the auxin perceptive proteins in the A431 pools. FACS sorting was performed to enrich A549-EGFR pools responsive to IAA treatment. For sorting, A549 pools were treated with 1 h IAA and sorted for cells with lower GFP.

Live Cell Airyscan Imaging

Cells cultured in FluoroBrite DMEM with 10% FBS in 8-well Lab-Tek II #1.5 coverglass slides (Thermofisher) were imaged with a Zeiss LSM 880 equipped with an Airyscan detector using a 63× Plan-apochromat oil objective NA 1.4. Live cell imaging was performed at 37° C., 5% CO₂ with incubator insert PM S1. Images were Airyscan processed automatically using the Zeiss Zen2 software package.

Analysis of Cell Division in A431 Cells with Tagged DHC1

Cells were plated on μ-slide 8-well ibiTreat dishes at 0.1×10⁵ cells per well 2 days before the experiment. On the experiment day, cells were loaded with 2 μM CellTracker™ Red CMTPX (Thermo, CAT #C34552) in complete medium for 15-30 min at 37° C. Medium was then changed to FluroBrite containing 10% FBS and incubated at 37° C. for 1-2 h before imaging to equilibrate the labelling. Cells were imaged with Nikon Eclipse Ti-E microscope equipped with 20× air objective, Nikon Perfect Focus System 3, Hamamatsu Flash 4.0 V2 scientific CMOS and Okolab stage top incubator system. Before recording, 6 fields for each of the 8 wells were selected with Celllracker™ Red fluorescence. IAA was then added at a final 0.5 mM concentration to IAA-treated cells, mixed well with pipetting, and time lapse imaging started immediately recording every 30 min for 16 h. Mitotic rounding cells were counted manually in the videos from 0.5 h to 6 h. Mitotic rounding cells without cell division were followed till 13 h.

EGF Uptake in A549 Cells with Tagged EGFR

A549 cells were seeded at 0.4×10⁵ cells per 4-well for 3 days. Medium was changed to fresh medium with or without 0.5 mM IAA for 1 h. Cells were washed twice with ice-cold serum free medium with 1% BSA and 0.2 ml of 2 μg/ml Alexa Fluor™ 647 EGF complex (ThermoFisher, E35351) in serum free medium with 1% BSA was added. Cells were further incubated at 37° C. for 20 min before harvesting with trypsin. Samples were kept on ice before FACS analysis. Alexa Fluor™ 647 was excited with 640 nm laser and detected with 720/40 detector, analysing 20 000 cells per sample. Negative control samples were cells incubated in medium without EGF complex. Background subtracted mean fluorescence intensity was used for analysis.

Glucose Uptake in A431 Cells with Tagged Glut1

Cells were plated at 0.6×10⁵ cells on 4-well plates 2 days prior the experiment. On the experiment day, cells were treated with or without 0.5 mM IAA for 1 h at 37° C., then washed with DPBS (Gibco, 14040117 with calcium, magnesium). Glucose uptake was measured by incubating cells with 1 mM 2-DG in DPBS for 10 min at RT and subsequent steps were performed according to the manufacturer's protocol (Promega, Cat #J1341). Luminescent signal was measured in a 96 black well microplate (SCREENSTAR, Cat #655866) with VICTOR X3 multimode plate reader (PerkinElmer). Cells incubated with DPBS only were used as background. Protein concentrations were measured with BioRad DC assay. Glucose uptake after background subtraction was normalized to protein concentration.

F-Actin Staining in A431 Cells with Tagged NMIIa

Cells were plated at 0.3×105 on μ-slide 8-well ibiTreat chambers 1 day before the experiment. On the experiment day, cells were treated with or without 0.5 mM IAA for 2 h at 37° C., then washed with PBS, fixed with 4% PFA in 250 mM Hepes, pH 7.4, 100 μM CaCl₂ and 100 μM MgCl₂ for 20 min, followed by quenching in 50 mM NH₄Cl for 10 min and 3 washes with PBS. Cells were then stained with 0.132 μM Alexa Fluor 568 Phalloidin (Molecular probes A-12380) in PBS for 30 min at RT. Z-stacks spanning the whole cell (step size 0.3 μm) were acquired with Nikon Eclipse Ti-E microscope, 60× PlanApo VC oil objective NA 1.4, with 1.5× zoom. Image stacks were automatically deconvolved using the Huygens batch processing application (Scientific Volume Imaging), and deconvolved image stacks were maximum intensity projected in ImageJ FIJI.

Filipin Staining in A431 Cells with Tagged NPC1

Cells on coverslips in complete medium were treated with or without 0.5 mM IAA for 16 h, fixed and quenched as above. Fixed cells were then stained with 50 μg/ml filipin in PBS for 30 min at 37° C. Cells were washed twice with PBS and mounted with mowiol-DABCO. Imaging was performed on a Nikon Eclipse Ti-E microscope equipped with 100× oil objective NA 1.4.

Lipid Droplet Biogenesis in A431 Cells with Tagged Seipin

Cells were delipidated by culturing in serum-free medium supplemented with 5% lipoprotein-deficient serum for 3 days and treated with or without 0.5 mM IAA for the final 16 h on μ-slide 8-well ibiTreat slides. For LD biogenesis, cells were loaded with 0.2 mM oleic acid (oleic acid prepared as 1 mM OA-BSA complex at 10:1 molar ratio to BSA in serum-free DMEM) for the final 2 h, fixed and quenched as above. Lipid droplets were stained with LD540 (synthesized by Princeton BioMolecular Research, 0.1 μg/ml) and nuclei with DAPI (Sigma, D9542, 10 μg/ml). Z-stacks spanning the whole cell (step size 0.3 μm) were acquired with Nikon Eclipse Ti-E microscope, 60× PlanApo VC oil objective NA 1.4, with 1.5× zoom lens, and image stacks were automatically deconvolved using the Huygens batch processing application (Scientific Volume Imaging), and deconvolved image stacks maximum intensity projected by custom MATLAB scripts. Cell segmentation, LD detection and LD size distribution analysis was performed with CellProfiler and custom MATLAB software generated for post-processing.

Cholesterol Measurement in A431 Cells with Tagged LBR

Cells were delipidated by culturing in serum-free medium supplemented with 5% lipoprotein-deficient serum for 3 days and treated with or without 0.5 mM IAA for the final 48 h. Cells were washed and harvested with ice-cold PBS. Cell pellets were used for measurement. Cholesterol was measured by gas-liquid chromatography (GLC) analysis. The chloroformmethanol extracts of cellular lipids were saponified with potassium hydroxide in ethanol, extracted with hexan, and silylated with trichloromethylsilane. Cholesterol was separated from noncholesterol sterols and squalene and quantified by capillary GLC with flame ionization detection and using a 50-m capillary column (Ultra 2; Agilent Technologies, Wilmington, Del., USA) with 5α-cholestane as the internal standard. Protein concentration was measured from an aliquot of the same samples with Bio-Rad DC Protein assay.

Western Blotting

Cells were lysed in buffer containing 1.0% Igepal CA-630, 0.5% sodium deoxycholate, 0.1% sodium dodecyl sulfate, 250 mM Tris-HCl, pH. 7.5 and 150 mM NaCl with protease inhibitors. Equal amounts of protein (measured using DC Protein assay) were loaded onto 12% Mini-Protean TGX Stain-Free gels and transferred onto LF PVDF membrane (Bio-Rad). Membranes were blotted with Odyssey blocking buffer (LI-COR) at RT for 0.5-1.0 h, incubated with first antibody (rabbit anti-GFP: ab290 Abcam; Mouse anti-alpha Tubulin: Sigma B-5-1-2; Mouse anti-PMP70: Sigma SAB4200181) at 4° C. overnight. Detection was performed with IRDye 800CW goat anti-mouse (Li-cor 926-32210) and Alexa 680 goat anti-rabbit antibodies (Invitrogen A21109) and images were acquired with a ChemiDoc Imaging System (BioRad).

Analysis of PMP22-mCardinal Fluorescence in A431 Cells with Tagged PEX3

Cells were transfected with mCardinal-PMP-N (Addgene plasmid #56173, a gift from Michael Davidson). Single clones with low mCardinal-PMP-N-10 expression and proper subcellular localization were isolated after FACS sorting of low mCardinal fluorescent cells. Cells were treated with or without 0.5 mM IAA for 14 days and seeded on p-slide 8-well ibiTreat chambers for the final 2 days. Cells were fixed and quenched as above. Nuclei were stained with DAPI (Sigma, D9542, 10 μg/ml). Z-stacks spanning the whole cell (step size 0.3 μm) were acquired with Nikon Eclipse Ti-E microscope, 60× PlanApo VC oil objective NA 1.4, with 1.5× zoom lens. Maximum intensity projections were generated in FIJI and cells segmented in CellProfiler as described above for LD analysis. Background subtracted PMP22-mCardinal fluorescence intensity was analyzed from the segmented images using a custom MATLAB software generated for post-processing.

### Example 3—Characterization of the New AID System with Molecular Dynamics Simulations

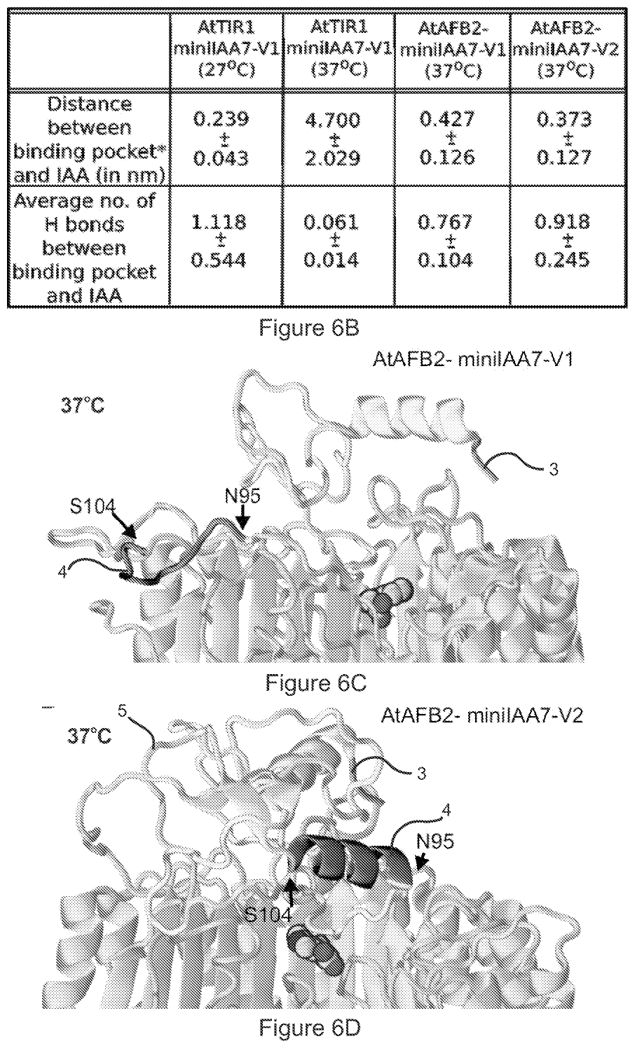

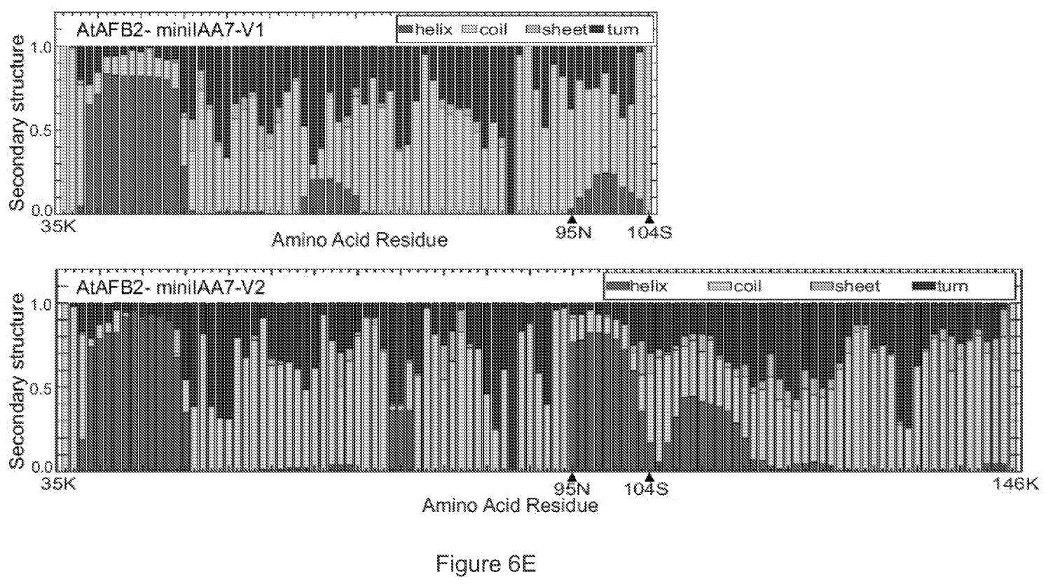

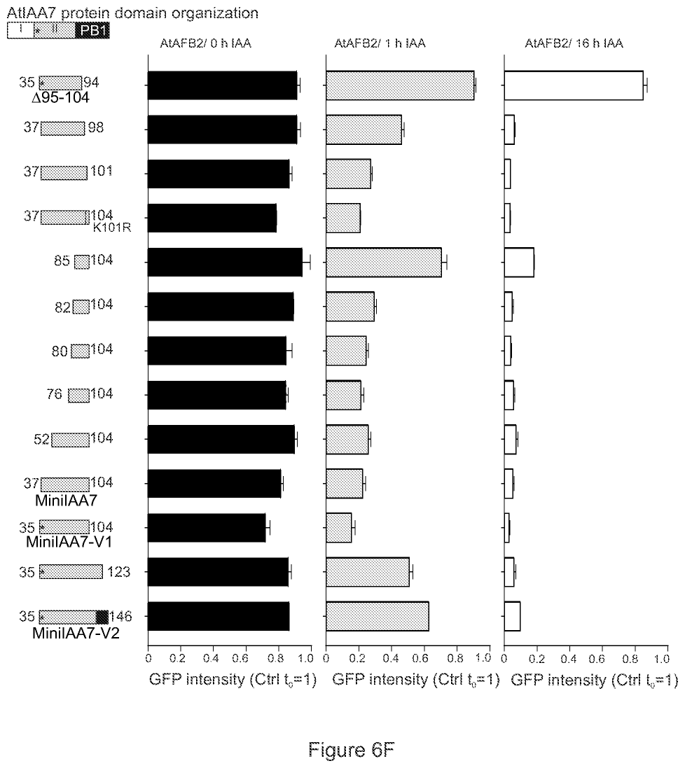

Finally, atomistic molecular dynamics simulations were conducted to gain insight into the AtAFB2-miniIAA7 system. The simulations revealed that the interactions between IAA and its binding pocket are substantially weaker in AtTIR1 compared to AtAFB2 at 37° C. (FIG. 6A, B). This is consistent with AtAFB2 working robustly in mammalian cells at 37° C. (the present Examples) and AtTIR1 only being functional at lower temperatures, as shown in yeast. Simulations of AtAFB2 with variants of miniIAA7 demonstrated profound secondary structure changes in aa. 95-104 of miniIAA7 (Aa. 95-104 of SEQ ID NO: 1). Aa. 95-104 represent a previously uncharacterized stretch. It adopts an alpha-helical structure when followed by an extended C-terminus but maintains a flexible coil structure when lacking the extension (FIG. 6C-E). The simulations thus suggest that the presence of the C-terminal segment (aa. 105-146) increases the structural stability of aa. 95-104, and this likely hampers IAA-inducible rapid target degradation (FIG. 6F). The simulations further predict the critical importance of aa. 95-104, which was confirmed by experiments showing ablation of auxin-inducible degradation upon its removal (FIG. 6F). Further refinement of the miniIAA7 degron revealed that aa.82-101 behave similarly to miniIAA7 (FIG. 6F).

FIGS. 6A and 6B show characterization of AtTIR1, AtAFB2 and miniIAA7 through atomistic molecular dynamics simulations. FIG. 6A, schematic representation, and FIG. 6B, table characterizing the amino acid residues of IAA binding pocket involved in IAA binding in AtTIR1 and AtAFB2 by simulations (n=5). AtTIR1 backbone is shown in the background as transparent. IAA is depicted in van der Waals representation. Residues defining IAA binding pockets are illustrated in blue/licorice representation, with AtTIR1 residues in darker blue (reference number 1) and AtAFB2 residues in lighter blue (2). Residue numbers refer to those of AtTIR1. Residues in larger font represent ones involved in interaction with IAA in the simulations and in the crystal structure (PDB ID: 2P1P), red residue numbers represent ones involved in IAA interaction in AtTIR1 but not in AtAFB2.

FIGS. 6C and 6D are representative snapshots highlighting miniIAA7-V1 and -V2 degrons in the indicated complexes at the end of 1 μs simulations (n=5). Magenta: N-terminal KR dipeptide (3); brown: aa. 95-104 (4); pink: C-terminal extension after 5104 (5).

Together, these findings emphasize the importance of maintaining structural flexibility of aa. 95-104 in miniIAA7 and help to explain why miniIAA7-mEFGP works as a fixed unit.

Atomistic Molecular Dynamics Simulations

Atomic co-ordinates for AtTIR1 were obtained from the protein data bank (PDB ID: 2P1P). The two AtIAA7 (SEQ ID NO: 1) peptide sequences used (miniIAA7-V1 and miniIAA7-V2) were modeled using multiple templates: aa. 35-81 had no homologous structure available and were modeled ab initio using the I-TASSER online software (for protein structure and function predictions c-score −1.45); aa. 82-94 were modeled based on the structure of the peptide in the crystal structure (PDB ID: 2P10); aa. 95-104 for miniIAA7-V1 and aa. 95-146 for miniIAA7-V2 were modeled on the solution NMR structure of a homologous protein IAA17 (PDB ID: 2MUK). AtTIR1, in complex with IAA, inositol hexakisphosphate (IHP), and miniIAA7-V1 was generated ensuring that the orientation of AtIAA7 (aa. 82-94) matched its crystal structure in complex with AtTIR1 (PDB ID: 2P10). The homology model of AtAFB2 was designed using the crystal structure of AtTIR1 as the template (PDB ID: 2P1P). A similar protocol was followed for obtaining AtAFB2 in complex with IAA, IHP and miniIAA7-V1 or AtAFB2 in complex with IAA, IHP and miniIAA7-V2. Stability of the homology models was validated based on the structural deviations from their initial conformation after simulation of these models for 200 ns. Further validation included comparison of simulation results and experiments described in this specification.

For simulations, the system was solvated in a box of 12×12×12 nm³ with KCl concentration of 150 mM. The CHARMM36 force field was used for proteins, IAA and IHP. Mol2 files for IAA and IHP were generated using Openbabel (O'Boyle, N. M. et al. J. Cheminform. 3, 33 (2011)) which were subsequently uploaded to Paramchem server (https://cgenff.umaryland.edu/) to obtain Toppar stream files (STR) for use with CgenFF, version 3.0.1 (Vanommeslaeghe, K. et al. J. Comput. Chem. 31, NA-NA (2009)). The STR files were converted to GROMACS topology file using cgenff_charmm2gmx.py script (http://mackerell.umaryland.edu/charmm ff.shtml). The TIP3P-CHARMM model was used for water. Simulations were performed using GROMACS 5.1.4 (Van Der Spoel, D. et al. J. Comput. Chem. 26, 1701-1718 (2005)). Each system was energy minimized. With position restraints applied on the protein, the system was simulated under constant NpT conditions using the V-rescale thermostat (Bussi et al., J. Chem. Phys. 126, 014101 (2007)) (300 K) and the Parrinello-Rahman barostat (Parrinello & Rahman, J. Appl. Phys. 52, 7182-7190 (1981)) (1 atm pressure isotropically applied along all dimensions) for 1 ns to allow solvent equilibration around the protein. A time step of 2 fs was used for integrating equations of motion. The LINCS algorithm (Hess, P-LINCS: A Parallel Linear Constraint Solver for Molecular Simulation, (2007), doi:10.1021/CT700200B) was employed to constrain the motions of covalently bonded hydrogen atoms. Neighbor list was updated using the Verlet cut-off scheme. A cut-off radius of 1 nm was applied to calculate van der Waals (Lennard-Jones) interactions, however the forces were smoothly switched to zero between 1.0 and 1.2 nm. Long-range electrostatic interactions (with a cut-off of 1.0 nm for the real-space component) were calculated using the Particle Mesh Ewald (PME) method (Darden et al., J. Chem. Phys. 98, 10089-10092 (1993)). Following equilibration, position restraints on the protein were removed and the simulations were continued for 1 μs. 5 replicate simulations with different initial conditions were carried out for each of the 4 systems.

To examine the interaction of IAA with auxin perceptive proteins, the average distance between the center-of-mass of backbone of binding pocket residues and IAA was estimated. Binding pocket was defined as residues in auxin perceptive proteins within 0.4 nm of IAA (taken from the initial conformation similar to that observed in the crystal structure PDB ID: 2P1P). The stability of IAA interaction also characterized by estimating the number of hydrogen bonds it formed with the residues of the binding pocket. Values were averaged over the entire simulation period and across all the replicas for all analyses.

Overall, these results demonstrate that the new AID system is suitable for loss-of-function studies to reveal both acute phenotypes (DHC1, Glut1 and MHY9; 0.5-2.0 h) and chronic phenotypes (Seipin and LBR; 16-48 h) with dramatic and specific IAA inducible changes. In addition, most of targets here are transmembrane proteins that have not been successfully depleted at the protein level using AID before, demonstrating that the new AID system is broadly applicable.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A method, a product, a system, or a use, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A polynucleotide comprising a nucleotide sequence encoding a degradation signal peptide, wherein the degradation signal peptide has an amino acid sequence comprising a sequence that is at least 75% identical to a sequence corresponding to amino acid residues
84-98 of SEQ ID NO: 1 (AtIAA7),
66-80 of SEQ ID NO: 2 (AtIAA3),
84-98 of SEQ ID NO: 3 (AtIAA17),
78-92 of SEQ ID NO: 4 (AtIAA14),
55-69 of SEQ ID NO: 5 (AtIAA5), or
167-181 of SEQ ID NO: 6 (AtIAA8),
or a degradation signal peptide functionally and/or structurally equivalent thereto.

2. The polynucleotide according to claim 1, wherein the degradation signal peptide comprises a sequence represented by formula I
X₁X₂VGWPPX₃X₄X₅X₆X₇X₈ Formula I
wherein
X₁ is Q or absent;
X₂ is absent, V, I, A, or L;
X₃ is V, I, L, G, or A;
X₄ is R, C, or K;
X₅ is N or S;
X₆ is Y, F, or W;
X₇ is R or K; and
X₈ is K or R;
optionally followed by a sequence represented by formula II
X₉X₁₀X₁₁X₁₂X₁₃X₁₄X₁₅X₁₆X₁₇X₁₈ Formula II
wherein
X₉ is N, S, T, K, or R;
X₁₀ is M, I, V, N, S, T, or L;
X₁₁ is M, I, L, V, S, or T;
X₁₂ is T, A, V, G, Q, H, S, L, F, or I;
X₁₃ is absent, Q, N, H, T, S, A, E, P, I, or L;
X₁₄ is absent, Q, P, C, S, Y, K, N, R, or T;
X₁₅ is absent, K, Q, T, P, S, N, or R;
X₁₆ is absent, S, N, T, K, P, or A; and
X₁₇ is absent, S, G, A, P, E, T, N, K, or R;
X₁₈ is absent, S, E, T, G, or N;
or a degradation signal peptide functionally and/or structurally equivalent thereto.

3. The polynucleotide according to claim 1, wherein the amino acid sequence of the degradation signal peptide ends at a residue corresponding to an amino acid residue in the range of amino acid residues
98-123 or 101-122 of SEQ ID NO: 1 (AtIAA7),
80-91 or 83-90 of SEQ ID NO: 2 (AtIAA3),
98-109 or 101-108 of SEQ ID NO: 3 (AtIAA17),
92-109 or 95-108 of SEQ ID NO: 4 (AtIAA14), 69-75 or 72-74 of SEQ ID NO: 5 (AtIAA5), or
181-198 or 184-197 of SEQ ID NO: 6 (AtIAA8).

4. The polynucleotide according to claim 1, wherein the amino acid sequence of the degradation signal peptide does not comprise a sequence starting at amino acid residues corresponding to
124 of SEQ ID NO: 1 (AtIAA7),
92 of SEQ ID NO: 2 (AtIAA3),
110 of SEQ ID NO: 3 (AtIAA17),
110 of SEQ ID NO: 4 (AtIAA14),
76 of SEQ ID NO: 5 (AtIAA5), or
199 of SEQ ID NO: 6 (AtIAA8).

5. The polynucleotide according to claim 1, wherein the amino acid sequence of the degradation signal peptide comprises or consists of a sequence
starting at an amino acid residue in the range of amino acid residues corresponding to amino acid residues at positions 1-84, or 1-83, or 1-82, or 1-81, or 35-83, or 35-82, or 35-81, of SEQ ID NO: 1 (AtIAA7), and ending at a residue corresponding to an amino acid residue in the range of amino acid residues at positions 98-123, or 99-123, or 100-123, or 101-122 of SEQ ID NO: 1 (AtIAA7);
starting at an amino acid residue in the range of amino acid residues corresponding to amino acid residues at positions 1-66, or 1-65, or 1-64, or 1-63, or 37-65, or 37-64, or 37-63, of SEQ ID NO: 2 (AtIAA3), and ending at a residue corresponding to an amino acid residue in the range of amino acid residues at positions 80-91, or 81-91, or 82-91, or 83-90 of SEQ ID NO: 2 (AtIAA3);
starting at an amino acid residue in the range of amino acid residues corresponding to amino acid residues at positions 1-84, or 1-83, or 1-82, or 1-81, or 31-83, or 31-82, or 31-81, of SEQ ID NO: 3 (AtIAA17), and ending at a residue corresponding to an amino acid residue in the range of amino acid residues at positions 98-109, or 99-109, or 100-109, or 101-108 of SEQ ID NO: 3 (AtIAA17);
starting at an amino acid residue in the range of amino acid residues corresponding to amino acid residues at positions 1-78, or 1-77, or 1-76, or 1-75, or 30-77, or 30-76, or 30-75, of SEQ ID NO: 4 (AtIAA14), and ending at a residue corresponding to an amino acid residue in the range of amino acid residues at positions 92-109, or 93-109, or 94-109, or 95-108 of SEQ ID NO: 4 (AtIAA14);
starting at an amino acid residue in the range of amino acid residues corresponding to amino acid residues at positions 1-55, or 1-54, or 1-53, or 1-52, or 34-54, or 34-53, or 34-52, of SEQ ID NO: 5 (AtIAA5), and ending at a residue corresponding to an amino acid residue in the range of amino acid residues at positions 69-75, or 70-75, or 71-75, or 72-74 of SEQ ID NO: 5 (AtIAA5);
starting at an amino acid residue in the range of amino acid residues corresponding to amino acid residues at positions 1-167, or 1-166, or 1-165, or 1-164, or 107-166, or 107-165, or 107-164 of SEQ ID NO: 6 (AtIAA8), and ending at a residue corresponding to an amino acid residue in the range of amino acid residues at positions 181-198, or 182-198, or 183-198, or 184-197 of SEQ ID NO: 6 (AtIAA8);
or a sequence at least 75%, or at least 80%, or at least 85%, or at least 90%, or at least 95%, or 100% identical thereto;
or a degradation signal peptide functionally and/or structurally equivalent thereto.

6. The polynucleotide according to claim 1, wherein the polynucleotide further comprises a sequence encoding a target polypeptide or protein or a moiety capable of associating with a target polypeptide or protein, such that the target polypeptide or protein or the moiety is fused to the degradation signal peptide, optionally via a linker sequence.

7. The polynucleotide according to claim 1, wherein the polynucleotide is operatively linked to one or more sequences for expression in a host cell and/or comprises one or more sequences for introducing the nucleotide sequence encoding the degradation signal peptide to a gene of a host genome, thereby fusing the nucleotide sequence encoding the degradation signal peptide to a target gene; wherein the host cell is an animal cell or a fungal cell and/or the host genome is an animal genome or a fungal genome.

8. The polynucleotide according to claim 1, wherein the polynucleotide and/or the nucleotide sequence encoding the degradation signal peptide is codon optimized for expression in a host cell, and wherein the host cell is an animal cell or a fungal cell.

9. A polypeptide or protein comprising the degradation signal peptide encoded by the nucleotide sequence encoding the degradation signal peptide of the polynucleotide according to claim 1.

10. The polypeptide or protein according to claim 9, wherein the polypeptide or protein is a fusion polypeptide or a fusion protein comprising the degradation signal peptide fused to a target polypeptide or protein or to a moiety capable of associating with a target polypeptide or protein, optionally via a linker sequence.

11. An expression cassette comprising the polynucleotide according to claim 1, wherein the expression cassette comprises one or more sequences for expression in a host cell, and the nucleotide sequence encoding the degradation signal peptide and/or the polynucleotide is operatively linked to the one or more sequences for expression in the host cell, and/or wherein the expression cassette comprises one or more sequences for introducing the nucleotide sequence encoding the degradation signal peptide and/or the polynucleotide to a host genome, optionally fusing the nucleotide sequence encoding the degradation signal peptide and/or the polynucleotide to a target gene; and wherein the host cell is an animal cell or a fungal cell.

12. A vector comprising the polynucleotide according to claim 1.

13. The vector according to claim 12, wherein the nucleotide sequence encoding the degradation signal peptide and/or the polynucleotide is operatively linked to one or more sequences for expression in a host cell, and/or wherein the vector comprises one or more sequences for introducing the nucleotide sequence encoding the degradation signal peptide and/or the polynucleotide to a host genome, thereby fusing the nucleotide sequence encoding the degradation signal peptide and/or the polynucleotide to a target gene; and wherein the host cell is an animal cell or a fungal cell.

14. A system for at least partially depleting a target polypeptide or protein in a host cell, the system comprising the polynucleotide according to claim 1, and
a second polynucleotide, a second expression cassette and/or a second vector comprising the second polynucleotide, wherein the second polynucleotide encodes a functional auxin perceptive protein capable of binding the degradation signal peptide in the presence of auxin or an auxin analogue;
wherein the host cell is an animal cell or a fungal cell.

15. The system according to claim 14, wherein the functional auxin perceptive protein is AtAFB2 (SEQ ID NO: 96) or a polypeptide or a protein comprising at least one stretch that is at least 80% identical to a continuous stretch of at least 60 amino acids of AtAFB2 (SEQ ID NO: 96).

16. The system according to claim 14, wherein the second polynucleotide, expression cassette and/or vector further comprise(s) a nucleotide sequence encoding a localization sequence for directing the localization of the functional auxin perceptive protein, such as a nuclear localization sequence.

17. A kit comprising the polynucleotide according to claim 1 and optionally instructions for use.

18. A host cell comprising the nucleotide sequence encoding the degradation signal peptide and/or the polynucleotide according to claim 1, wherein the host cell is an animal cell or a fungal cell.

19. The host cell according to claim 18, wherein the host cell is a mammalian cell, for example a human, murine, bovine, ovine, porcine, feline, canine, equine, or primate cell; a nematode cell; or an insect cell.

20. A transgenic organism stably transformed or transfected with the nucleotide sequence encoding the degradation signal peptide and/or the polynucleotide according to claim 1.

21. A method for at least partially depleting a target polypeptide or protein in a host cell, the method comprising
introducing the polynucleotide according to claim 1 to the host cell, such that the nucleotide sequence encoding the degradation signal peptide and/or the polynucleotide forms a fusion with a target gene encoding the target polypeptide or protein or a moiety capable of associating with the target polypeptide or protein, the fusion encoding a fusion protein comprising the degradation signal peptide and the target polypeptide or protein or the moiety capable of associating with the target polypeptide or protein; or providing the host cell, wherein the nucleotide sequence encoding the degradation signal peptide and/or the polynucleotide forms a fusion with a target gene encoding the target polypeptide or protein or a moiety capable of associating with the target polypeptide or protein, the fusion encoding a fusion protein comprising the degradation signal peptide and the target polypeptide or protein or the moiety capable of associating with the target polypeptide or protein;
expressing the fusion protein in the host cell;
expressing a functional auxin perceptive protein in the host cell; and
introducing auxin or an auxin analogue to the host cell, such that the auxin or the auxin analogue binds to the functional auxin perceptive protein and induces at least a partial depletion of the fusion protein or of the target polypeptide or protein by causing the auxin perceptive protein to bind to the degradation signal peptide;
wherein the host cell is an animal cell or a fungal cell.

22. The method according to claim 21, wherein the functional auxin perceptive protein is AtAFB2 (SEQ ID NO: 96) or a polypeptide or a protein comprising at least one stretch that is at least 80% identical to a continuous stretch of at least 60 amino acids of AtAFB2 (SEQ ID NO: 96).

23. A method for producing a host cell comprising introducing the polynucleotide according to claim 1 into the host cell, wherein the host cell is an animal cell or a fungal cell.

24. (canceled)
